(19) United States
(12) Patent Application Publication
(10) Pub. No.: US 2009/0191543 A1
(43) Pub. Date: Jul. 30, 2009

(54) IDENTIFICATION OF RAC1B AS A MARKER AND MEDIATOR OF METALLOPROTEINASE-INDUCED MALIGNANCY

(75) Inventors: Derek C. Radisky, Jacksonville, FL (US); Celeste M. Nelson, Princeton, NJ (US); Mina J. Bissell, Berkeley, CA (US)

Correspondence Address: LAWRENCE BERKELEY NATIONAL LABORATORY, Technology Transfer & Intellectual Propery Managem, One Cyolotron Road MS 56A-120, BERKELEY, CA, 94720, US

(73) Assignee: THEREGENTS OF THE UNIVERSITY OF CALIFORNIA, Oakland, CA (US)

(21) Appl. No.: 11/915,726

(22) PCT Filed: May 26, 2006
(86) PCT No.: PCT/US06/20467

§ 371 Date: Dec. 10, 2007

Related U.S. Application Data
(60) Provisional application No. 60/685,428, filed on May 27, 2005.

(51) Int. Cl. C12Q 1/08; C12N 5/02; G01N 33/574; C07H 21/04; C12N 5/08

(52) U.S. Cl. 435/6; 435/375; 435/366; 435/7.23; 536/23.1

(57) ABSTRACT

The present invention provides compositions and methods for detecting MMP-induced malignancies by detecting Rac1b expression. The invention further provides compositions and in vitro and in vivo methods for inhibiting MMP-induced malignant transformation by modulating Rac1b expression and/or function.

### CROSS-REFERENCE TO RELATED APPLICATIONS

This application is the U.S. National Phase Entry of PCT Application No. PCT/US06/20467 filed 26 May 2006, which claimed the benefit of U.S. Provisional Patent Application No. 60/685,428, filed 27 May 2005, both of which are hereby incorporated by reference in their entirety for all purposes.

### STATEMENT OF GOVERNMENTAL SUPPORT

This invention was made with government support under Contract No. DE-AC03-76SF00098, now Contract No. DE-AC02-05CH11231 awarded by the U.S. Department of Energy. This work was also supported by grants from the OBER office of the Department of Energy and an Innovator award from the Department of Defense and from the National Institutes of Health, and by fellowships from the National Cancer Institute and the Department of Defense. The government has certain rights in this invention.

### FIELD OF THE INVENTION

The present invention relates to cancer markers and therapeutics. More specifically, the present invention relates to the detection of an early cancer marker to prevent epithelial-mesenchymal transition and genomic instability which can lead to malignant transformation of cells.

### BACKGROUND OF THE INVENTION

Cancer is characterized by a progressive series of alterations that disrupt cell and tissue homeostasis. Whereas many of these alterations can be induced by specific mutations, faulty signals from the microenvironment also can act as inducers of tumor development and progression (Bissell, M. J. & Radisky, D. Putting tumours in context. Nat Rev Cancer 1, 46-54, 2001). Matrix metalloproteinases (MMPs) are prominent contributors to such microenvironmental signals. MMPs are proteolytic enzymes that degrade structural components of the extracellular matrix (ECM), allowing for tumor invasion and metastasis. Additionally, MMPs can release cell-bound inactive precursor forms of growth factors, degrade cell-cell and cell-ECM adhesion molecules, activate precursor zymogen forms of other MMPs, and inactivate inhibitors of MMPs and other proteases (Egeblad, M. & Werb, Z. New functions for the matrix metalloproteinases in cancer progression. Nat Rev Cancer 2, 161-74, 2002). MMPs have been shown to be a causative factor in number of cancers including, e.g., cancers of the lung, breast, colon, skin, prostate, ovary, pancreas, uroepithelial cells, squamous cells, tongue, mouth, and stomach.

Due to the role of MMP in tumorigenesis and metastasis, compositions and methods for treating and preventing cancer by specifically targeting MMP's have been explored. However, attempts to treat or prevent cancer by directly inhibiting MMP's have not been successful in the clinic. Cancer patients receiving MMP inhibitors experienced a number of deleterious side effects (e.g., inflammation and acute pain) that led to cessation of the clinical trials and/or administration of drastically reduced doses of the MMP inhibitors in subsequent phases of the clinical trials (Coussens et al., Science 295: 2387, 2002).

Thus, there is a need in the art for compositions and methods for detecting expression of proteins that play a role in MMP-induced malignant transformation as well as methods and composition for modulating proteins that play a role in MMP-induced malignancy. The present invention satisfies these and other needs.

### SUMMARY OF THE INVENTION

The present invention provides compositions and methods for detecting Rac1b. The invention further provides compositions and methods for modulating expression of Rac1b.

One embodiment of the invention provides methods for inhibiting matrix metalloproteinase (MMP) induced malignant transformation of a cell, including, e.g., MMP-3 or MMP-9 induced malignant transformation of a cell. The method comprises contacting cell with a compound that modulates Rac1b. In some embodiments, the compound comprises an siRNA molecule (e.g., a molecule comprising a sequence selected from SEQ ID NOS: 1, 2, 3, and 4) that selectively inhibits expression of Rac1b. In some embodiments, the compound comprises an antibody (e.g., a monoclonal antibody, a humanized antibody, or an antibody fragment such as a (Fab′₂) fragment, a Fab fragment, a Fv fragment, or a scFv) that specifically binds to Rac1b. In some embodiments, the antibody specifically binds to a polypeptide encoded by a sequence selected from SEQ ID NOS: 5, 8 and subsequences thereof or to a polypeptide comprising a sequence selected from SEQ ID NOS: 6, 9, and subsequences thereof. In some embodiments, the cell is in a mammal including a rodent such as a mouse or a rat or primate such as a human, a chimpanzee, or a monkey). In some embodiments, the mammal is a human diagnosed with MMP-associated cancer (e.g., breast cancer, lung cancer, prostate cancer, pancreatic cancer, ovarian cancer, metastatic melanoma, uroepithelial cancer, invasive oral cancer, gastric cancer, and head and neck squamous cell carcinoma).

Another embodiment of the invention provides methods for detecting MMP induced malignancy by detecting expression of Rac1b, said method comprising detecting the sequence set forth in SEQ ID NOS: 5, 6, 8, 9 or a subsequence thereof. In some embodiments, the detecting comprises: (a) contacting a sample with an oligonucleotide that selectively hybridizes to a nucleic acid sequence selected from the group consisting of: SEQ ID NOS: 5, 8 and subsequences thereof under conditions sufficient for the oligonucleotide to form a complex with the sequence; (b) determining whether a complex forms between the oligonucleotide and the sequence; and (c) detecting expression of Rac1b by detecting the complex of step (b), whereby expression of Rac1b detects the MMP induced malignancy. In some embodiments, the detecting comprises: (a) contacting a sample with primers that specifically amplify a nucleic acid sequence comprising a sequence selected from the group consisting of: SEQ ID NOS: 5, 8 and subsequences thereof, under conditions sufficient to amplify the sequence; (b) determining whether an amplification product is formed; and (c) detecting expression of Rac1b by detecting the amplification product of step (b), whereby expression of Rac1b detects the MMP-3 induced malignancy. In some embodiments, the sample is from a mammal (e.g. a mouse, rat, or human) suspected of having MMP induced cancer (e.g., breast cancer, lung cancer, prostate cancer, pancreatic cancer, ovarian cancer, metastatic melanoma, uroepithelial cancer, invasive oral cancer, gastric cancer, and head and neck squamous cell carcinoma). In some embodiments, the detecting comprises (a) contacting a sample with an antibody that specifically binds to a polypeptide comprising a sequence selected from the group consisting of: SEQ ID NO: 6, 9, and subsequences thereof under conditions sufficient for the antibody form a complex with the polypeptide, (b) determining whether a complex forms between the antibody and the polypeptide; and (c) detecting expression of Rac1b by detecting the complex of step (b), whereby expression of Rac1b detects the MMP induced malignancy. In some embodiments, the detecting comprises (a) contacting a sample with an antibody that specifically binds to a polypeptide comprising a sequence encoded by a sequence selected from the group consisting of: SEQ ID NO: 5, 8, and subsequences thereof, under conditions sufficient for the antibody form a complex with the polypeptide, (b) determining whether a complex forms between the antibody and the polypeptide; and (c) detecting expression of Rac1b by detecting the complex of step (b), whereby expression of Rac1b detects the MMP induced malignancy. In some embodiments, the sample is from a mammal (e.g. a rat, mouse, or human) suspected of having MMP induced cancer (e.g., breast cancer, lung cancer, prostate cancer, pancreatic cancer, ovarian cancer, metastatic melanoma, uroepithelial cancer, invasive oral cancer, gastric cancer, and head and neck squamous cell carcinoma).

A further embodiment of the invention provides isolated nucleic acids comprising a sequence set forth in SEQ ID NOS: 1, 2, 3, or 4.

### BRIEF DESCRIPTION OF THE DRAWINGS

FIGS. 1A-H. MMP-3 induces EMT through Rac1b. a, MMP-3-induced alterations in actin cytoskeleton; scale bar, 25 μm. b, Analysis of active and total levels of Rac. c, RT/PCR of Rac1 and Rac1b. d, Rac1b protein expression. e, Rac1b transcript levels in response to MMP-3 treatment (days 1-4) and washout (days 5-6); blue diamonds, treated; red squares, untreated; p<0.001 for day 4 treated vs either day 1 treated or day 4 untreated. f, Cell motility assessed by scratch assay. g, Quantification of knockdown of endogenous gene expression; p<0.005 for untreated vs MMP-3, p<0.05 for untreated vs V12 (ca), p<0.005 for MMP-3 vs MMP-3+N17 (dn), p<0.001 for untreated vs Rac1b. h, Selective knockdown of Rac1b inhibits MMP-3-induced cell scattering. Scale bar, 25 μm. For all graphs, error bars represent SEM. p<0.05 for: Rac3/Rac3 siRNA vs Rac3/no siRNA, Rac3/Rac1 siRNA, or Rac3/Rac1b siRNA; Rac1/Rac1 siRNA vs Rac1/no siRNA, Rac1/Rac3 siRNA, or Rac1/Rac1b siRNA; and Rac1b/Rac1 siRNA or Rac1b/Rac1b siRNA vs Rac1b/no siRNA or Rac1b/Rac3 siRNA.

FIGS. 2A-G. MMP-3/Rac1b stimulate mitochondrial production of ROS. a, Cellular ROS levels assessed by DCFDA; error bars, SEM. p<0.005 for untreated vs MMP-3-treated or Rac1b; p<0.01 for MMP-3-treated vs MMP-3+Rac1N17. b, Mitochondrial pattern of DCFDA fluorescence; scale bar, 25 μm. c, Precipitation of nitrobluetetrazolium; scale bar, 15 μm. d, Mitochondrial depolarization assessed with JC-1; scale bar, 50 μm. e-g, Cells cotransfected with EYFP and either catalase (CAT; e), superoxide dismutase-1 (SOD1; f), or superoxide dismutase-2 (SOD2; g) and then cultured in the absence (upper image) or presence (lower image) of MMP-3 for 6 days. EYFP fluorescence, green; nuclei, red; graphs at bottom show gene transcript levels in transfected cell populations; scale bar, 100 μm.

FIGS. 3A-H. MMP-3-induced EMT is dependent upon ROS. a, NAC inhibits MMP-3-induced downregulation of epithelial cytokeratin protein levels; p<0.01 for MMP-3+NAC vs MMP-3 alone. b, Induction of Snail by MMP-3, and ROS dependence; p<0.001 for either untreated or MMP-3+NAC vs either MMP-3 or H2O2. c, Snail transcript levels in response to MMP-3 treatment (days 1-4) and washout (days 5-6); blue diamonds, treated; untreated, red squares. p<0.01 for treated days 4 vs either day 1 treated or day 4 untreated. d-e, Exogenous expression of Snail in SCp2 cells reduces E-cadherin transcript (p<0.01 for difference) (d) and protein levels (e). f, Cell scattering induced by treatment with MMP-3 or H₂O₂, or by exogenous expression of Snail; scale bar, 50 μm. g-h, ROS- and Snail-dependence of vimentin (g) and Rac1b (h) expression. For all graphs, error bars represent SEM. p<0.01 for either untreated or MMP-3+NAC vs either MMP-3, H₂O₂, or Snail (g), and for either untreated, H₂O₂, or Snail vs either MMP-3 or MMP-3+NAC (h).

FIGS. 4A-D. MMP-3-induced ROS cause DNA damage and genomic instability. a-b, 8-oxoguanosine induced treatment with MMP-3 (a; scale bar, 50 μm); quantification of increased nuclear staining relative to untreated (b; error bars, 95% CI, p<0.001 for MMP-3 vs. all other conditions). c, Induction of PALA resistance by MMP-3 (blue diamonds, MMP-3; red squares, untreated; p<0.05 for day 7, p<0.01 for days 14 and 28). d, Fluorescence in situ hybridization of CAD gene locus (red). e, ROS and oxygen dependence of PALA resistance induced by 14 d treatment with MMP-3. f, Frequency plots of CGH analyses of cells grown in the absence (top) or presence (bottom) of MMP-3, and then selected with PALA. p<0.01 for MMP-3 vs either untreated, MMP-3+NAC, or MMP-3 (3% O2), and p<0.005 for H₂O₂ vs untreated.

FIG. 5A-C. Properties of MMP-3-induced EMT. a, MMP-3-treated SCp2 cells, stained for cytokeratins (red), vimentin (green), and DNA (blue); scale bar, 50 μm. b, Marker transcript levels in cells treated with MMP-3 for 4 days; p<0.01 for all altered expression levels. c, Vimentin transcript levels in response to MMP-3 treatment (days 1-4) and washout (days 5-6); blue diamonds, treated; red squares, untreated; p<0.001 for day 4 treated vs either day 1 treated or day 4 untreated

FIGS. 6A-B. Dependence of MMP-3-induced EMT on Rac1 activity. Rac1-dependence was tested using tetracycline-regulated adenoviral expression vectors and a vimentin promoter reporter system (courtesy C. Gilles, University of Liege, Belgium). Activation of vimentin promoter by treatment with MMP-3 (4 d) is attenuated by inducible expression of dominant negative (dn) Rac1N17 (a), whereas inducible expression of constitutively active (ca) Rac1V 12 (4 d) is sufficient to activate vimentin promoter even in the absence of MMP-3 (b); insets show sample images of indicated experiments (green, GFP; red, nuclei).

FIG. 7. Selective knockdown of cotransfected constructs by siRNA. Selective knockdown of cotransfected constructs by siRNA. Insets, phase contrast images of upper right corner of the same field; scale bar, 25 μm.

FIGS. 8A-B. Induction of EMT by proteolytic activity of MMP-3. a, Catalytically inactive MMP-3 (MMP-3EA) does not induce EMT, and does not block EMT induced by active MMP-3. Scale bar, 50 μm. Graph, MMP-3EA expression in uninduced and induced cells, analyzed by quantitative RT/PCR and normalized to GAPDH expression. Error bars, SEM; p<0.001 for comparison. b, Activation of vimentin-EGFP construct and effect of MMP inhibitor (GM6001) on cells treated with MMP-3. Scale bar, 50 μm.

FIG. 9. Validation of Rac1b antibody. Cells were transfected with plasmids expressing YFP, cloned mouse Rac1b, YFP-Rac1b, or YFP-Rac1. Cell lysates were westernblotted using anti-Rac1 antibody (1:1000, Upstate), or the rabbit antisera raised against the Rac1b insert peptide (1:100, Biosource). Note that Rac1 antibody cross-reacts with Rac1b, but that the Rac1b antibody does not recognize Rac1.

FIG. 10. Effect of YFP-fused Rac1 and Rac1b constructs on cell morphology. Mouse Rac1b was cloned from cDNA derived from MMP-3-treated cells expressed as a fusion with YFP; endogenous mouse Rac1 was also cloned and used to generate active YFPRac1V12 and inhibitory YFPRac1N17 constructs. Left, Texas redphalloidin; right, YFP; scale bar, 25 μm.

FIG. 11. Activity assay of YFP-fused mouse Rac1b and Rac1V12.

### BRIEF DESCRIPTION OF THE SEQUENCES

SEQ ID NO: 1 sets forth an siRNA sequence that specifically inhibits Rac1b expression.

SEQ ID NO: 2 sets forth an siRNA sequence that specifically inhibits Rac1b expression.

SEQ ID NO: 3 sets forth an siRNA sequence that specifically inhibits Rac1b expression.

SEQ ID NO: 4 sets forth an siRNA sequence that specifically inhibits Rac1b expression.

SEQ ID NO: 5 sets forth the nucleotide sequence for the Rac1b insertion,

SEQ ID NO: 6 sets forth the polypeptide sequence for the Rac1B insertion.

SEQ ID NO: 7 sets forth the nucleotide sequence for human Rac1 cDNA.

SEQ ID NO: 8 sets forth the nucleotide sequence for human Rac1b cDNA.

SEQ ID NO: 9 sets forth a polypeptide sequence used to generate an antibody that specifically binds to Rac1b.

SEQ ID NO: 10 sets forth an enzymatic cleavage sequence.

SEQ ID NO: 11 sets forth a FITC-avidin staining blocking oligonucleotide.

SEQ ID NO: 12 sets forth a FITC-avidin staining control oligonucleotide.

### DETAILED DESCRIPTION OF THE INVENTION

### I. Introduction

The invention is based on the discovery that the Rho GTPase, Rac1b plays a role in MMP (e.g. MMP-3 and MMP-9) induced malignant transformation of cells. Prior to the studies described here, no information was available concerning the specific role of Rac1b in tumor progression, nor was any information available concerning the physiological mechanisms involved in the induction of Rac1b. Specifically, the present inventors have discovered that MMP (e.g., MMP-3 and MMP-9) induces expression of Rac1b which in turn induces an increase in the level of cellular reactive oxygen species (ROS). ROS stimulate expression of the transcription factors Snail and EMT which in turn cause oxidative damage and genomic instability leading to malignant transformation of cells.

The invention provides compositions and methods for modulating Rac1b expression. The compositions and methods are useful for preventing malignant transformation of cells and for treating disease and disorders such as cancer (e.g., MMP induced cancer). The invention further provides compositions and methods for detecting Rac1b expression. The compositions and methods are useful for diagnosis and prognosis of malignant disorders (e.g., MMP induced cancer). The compositions and methods can also be used to identify compounds useful for treating such disorders.

### II. Definitions

“Rac1b” refers to a splice variant of the Rho GTPase, Rac1, that contains a 57 nucleotide in-frame insertion that results in a 19 amino acid insertion. Rho GTPases bind and hydrolyze GTP; when in the GTP-bound state, they interact with effector proteins and modulate cell function. Rac1b is a highly activated isoform of Rac1, and has been found in tumors of the colon (Jordan, P., Brazao, R., Boavida, M. G., Gespach, C. & Chastre, E. Cloning of a novel human Rac1b splice variant with increased expression in colorectal tumors. Oncogene 18, 6835-9, 1999). Rac1b has been expressed in recombinant form and found to be highly activated (Matos, P., Collard, J. G. & Jordan, P. Tumor-related alternatively spliced Rac1b is not regulated by Rho-GDP dissociation inhibitors and exhibits selective downstream signaling. J Biol Chem 278, 50442-8, 2003; Fiegen, D. et al. Alternative splicing of Rac1 generates Rac1b, a self-activating GTPase. J Biol Chem 279, 4743-49, 2004), and to have transforming properties when expressed in fibroblast cultured cells (Singh, A. et al. Rac1b, a tumor associated, constitutively active Rac1 splice variant, promotes cellular transformation. Oncogene 23, 9369-80, 2004).

“MMP” or “matrix metalloproteinase” refers to zinc-dependent endopeptidases. MMPs degrade a variety of extracellular matrix proteins and process a number of bioactive molecules. For example, MMPs are known to be involved in the cleavage of cell surface receptors, the release of apoptotic ligands (such as the FAS ligand), and chemokine in/activation. MMPs are also thought to play a major role on cell behaviors such as cell proliferation, migration (adhesion/dispersion), differentiation, angiogenesis, apoptosis and host defense.

“MMP-3” or “matrix metalloproteinase 3” refers to a proteoglycanase closely related to collagenase (MMP1) with a wide range of substrate specificities. MMP-3 is a secreted metalloprotease produced predominantly by connective tissue cells. Together with other metalloproteases, MMP-3 can synergistically degrade the major components of the extracellular matrix (Sellers and Murphy, Int. Rev. Connect. Tissue Res. 9: 151-190, 1981). MMP-3 is capable of degrading proteoglycan, fibronectin, laminin, and type IV collagen.

“MMP-9” or “matrix metalloproteinase 9” refers to a 92-kD type IV collagenase which is a secreted zinc metalloproteases. In mammals, MMP-9 degrades the collagens of the extracellular matrix. MMP-9 is produced by normal alveolar macrophages and granulocytes.

“Cancer” or “malignancy” as used herein refers to diseases or disorders characterized by aberrant or uncontrolled cell division. Cancers and malignancies include, e.g., solid tumors, non-solid tumors, and hematological malignancies. Cancers and malignancies includes primary tumors as well as metastatic tumors.

An “MMP-induced cancer” or “MMP-induced malignancy” as used herein refers to cancers or malignancy that are the result of MMP overexpression. Such cancers include, e.g. cancers of the lung, breast, colon, skin (e.g. melanoma and metastatic melanoma), prostate, ovary, pancreas, uroepithelial cells, squamous cells (e.g., head and neck squamous cell carcinoma), tongue, mouth (e.g., invasive oral cancer, and stomach (e.g., gastric tumors) (see, e.g. Ghilardi et al., Clin Cancer Res. 8 (12): 3820-3 (2002); Nakopoulou et al., Hum Pathol. 30 (4): 436-42 (1999); Heppner et al., Am. J. Pathol. 149 (1): 273-82 (1996); Iwata et al., Jpn. J. Cancer Res. 87 (6): 602-11 (1996); Rahko et al., Anticancer Res. 24 (6): 4247-53 (2004); Ranuncolo et al., Int. J. Cancer 106 (5): 745-51 (2003); Wang et al., Surgery 132 (2): 220-5 (2002); Ahmad et al., Am. J. Pathol. 152 (3): 721-8 (1998); Davies et al., Br. J. Cancer 67 (5): 1126-31 (1993); Nielsen et al., Lab Invest 77 (4): 345-55 (1997); Nikkola, et al., Int J Cancer 97 (4): 432-8 (2002); Nikkola et al., Clin Cancer Res 11 (14): 5158-66 (2005); Jung et al., Int J Cancer 74 (2): 220-3 (1997); Cardillo et al., Anticancer Res., 26 (2A): 973-82 (2006); Zhang et al., Prostate Cancer Prostatic Dis. 7 (4): 327-32 (2004); Ishimaru et al., Acta Oncol., 41 (3): 289-96 (2002); Wood et al., Clin. Exp. Metastasis 15 (3): 246-58 (1997); Hamdy et al., Br. J. Cancer 69 (1): 177-82 (1994); Smolarz et al., Pol. J. Pathol. 54 (4): 233-8 (2003); Davidson et al., Clin. Exp. Metastasis 17 (10): 799-808 (1999); Liokumovich et al., J. Clin. Pathol. 52 (3): 198-202 (1999); Gotlieb et al., Gynecol. Oncol., 82 (1): 99-104 (2001); Davidson et al., Mol. Cell Endocrinol. 187 (1-2): 39-45 (2002); Davidson et al., Cancer Metastasis Rev., 22 (1): 103-15 (2003); Ozalp et al., Eur. J. Gynaecol. Oncol. 24 (5): 417-20 (2003); Demeter et al., Anticancer Res., 25 (4): 2885-9 (2005); Bramhall et al., Br. J. Cancer 73 (8): 972-8 (1996); Yamamoto et al., J. Clin. Oncol. 19 (4): 1118-27 (2001); Gress et al., Int J Cancer 62 (4): 407-13 (1995); Maatta et al., Clin. Cancer Res. 6 (7): 2726-34 (2000); Kuniyasu et al., Clin. Cancer Res. 5 (1): 25-33 (1999); Gohji et al. Cancer 78 (11): 2379-87 (1996); Ozdemir et al. J Urol., 161 (4): p. 1359-63 (1999); Ozdemir et al., J Urol. 158 (1): 206-11 (1997); Monier et al., Eur. Urol. 42 (4): 356-63 (2002); Wiegand et al., Cancer 104 (1): 94-100 (2005); Impola et al., J. Pathol. 202 (1): 14-22 (2004); Riedel et al., Int. J. Oncol., 17 (6): 1099-105 (2000); Kubben et al., Br. J. Cancer 94 (7): 1035-40 (2006); Sier et al., Br. J. Cancer 74 (3): 413-7 (1996); Matsumura et al., J. Cancer Res. Clin. Oncol. 131 (1): 19-25 (2005); Fang et al., Carcinogenesis 26 (2): 481-6 (2005); Michael et al., J. Clin. Oncol. 17 (6): 1802-8 (1999); Bodey et al. In Vivo 15 (1): 65-70 (2001); Cox et al., Clin. Cancer Res. 6 (6): 2349-55 (2000); Edwards et al., Br. J. Cancer 88 (10): 1553-9 (2003); Herbst et al., Clin. Cancer Res. 6 (3): 790-7 (2000); Passlick et al., Clin. Cancer Res. 6 (10): 3944-8 (2000); Ylisirnio et al., Clin. Cancer Res. 7 (6): 1633-7 (2001); and Ylisirnio et al., Anticancer Res., 20 (2B): 1311-6 (2000)).

“Sample” or “biological sample” include sections of tissues such as biopsy (e.g., from tissue suspected of being malignant) and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g. guinea pig, rat, mouse; rabbit; or a bird; reptile; or fish.

“RNAi molecule” or an “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

The term “antibody” refers to a polypeptide encoded by an immunoglobulin gene or functional fragments thereof that specifically binds and recognizes an antigen (e.g., Rac1b).

The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology, Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of antibodies, e.g. recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3.sup.rd ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g. U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g. McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g. Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety (e.g. toxins). In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a Rac1b protein, polymorphic variants, alleles, orthologs, and conservatively modified variants, or splice variants, or portions thereof, can be selected to obtain only those polyclonal antibodies that are specifically immunoreactive with Rac1b proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g. hydroxyproline, y-carboxyglutamate, and O-phosphoserine. Amino acid analogs refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 6 C, with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

An “amplification reaction” refers to any chemical reaction, including an enzymatic reaction, which results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20 (7):1691 (1992); Walker PCR Methods Appl 3 (1):1 (1993)), transcription-mediated amplification (Phyffer, et al, J. Clin. Microbiol. 34:834 (1996); Vuorinen, et al., J. Clin. Microbiol. 33:1856 (1995)), nucleic acid sequence-based amplification (NASBA) (Compton, Nature 350 (6313):91 (1991), rolling circle amplification (RCA) (Lisby, Mol. Biotechnol. 12 (1):75 (1999)); Hatch et al., Genet. Anal. 15 (2):35 (1999)) and branched DNA signal amplification (bDNA) (see, e.g., Iqbal et al., Mol. Cell Probes 13 (4):315 (1999)).

The terms “effective amount” or “amount effective to” or “therapeutically effective amount” refers to an amount sufficient to induce a detectable therapeutic response in the subject. Preferably, the therapeutic response is effective in inhibiting malignant transformation of a cell, including for example a cancer cell such as a cancer cell from an MMP (e.g. MMP-3 or MMP-9) induced malignancy.

### III. Detection of Rac1b Expression

In one embodiment, the invention provides methods for detecting MMP (e.g., MMP-3 OR MMP-9) induced malignant transformation by detecting Rac1b expression. Detection of Rac1b expression can be used in diagnostic and prognostic methods as well as in development of therapeutic compounds and methods. For example, Rac1b expression can be detected in samples from individuals suspected of having MMP induced cancer (e.g., cancer of the lung, breast, skin, prostate, ovary, pancreas, uroepithelial cells, squamous cells, tongue, mouth, and stomach), thus providing information regarding the likelihood that the potentially cancerous cells will undergo a malignant transformation. In some embodiments, Rac1b expression is detected following contacting a cell with a test compound to determine the effect of the test compound on Rac1b expression. Such information can be used, e.g. to identify and develop compounds useful for modulating Rac1b expression.

### A. Fluorescence in situ Hybridization

In some embodiments, elevated Rac1b expression is detected using fluorescence in situ hybridization (FISH) to detect Rac1b amplification. For example, probes that hybridize to the 57 nucleotide insertion region of Rac1b, i.e., SEQ ID NO: 5 ttg gag aca cat gtg gta aag ata gac cct cga ggg gca aag aca agc cga ttg ccg, can be developed.

In another embodiment, probes can be created by methods known in the art further based upon the 19 unique amino acid isoform sequence of Rac1b, SEQ ID NO: 6.

| | |
|---|---|
| SEQ ID NO: 6 | VGDTCGKDRPSRGKDKPI |

DNA from the probe generated can be produced and labeled by companies, such as Vysis, Inc., with known fluorescent dyes, such as Spectrum Orange, Spectrum Green and Spectrum Aqua to produce hybridization probes for detection of amplification at the test loci. In a preferred embodiment, probe production and labeling will be accomplished using Good Manufacturing Practices (GMP) at Vysis so that the analyses will be useful in obtaining FDA approval for clinical use of these markers.

In another embodiment, elevated Rac1b expression is detected using FISH to detect Rac1b amplification based upon genomic sequence containing and flanking Rac1 in GenBank Accession Nos. NT_{—}007819, NT_{—}086702, and NT_{—}079592, which are hereby incorporated by reference. Rac1 is located on chromosome 7p22, and the Unigene Locus number is Hs.413812. Other useful sequences for making probes and other sequences in the present invention include but are not limited, human Rac1 cDNA found at GenBank Accession No. NM_{—}006908 (SEQ ID NO: 7), and human Rac1b cDNA found in GenBank Accession No. NM_{—}018890 (SEQ ID NO: 8) which are hereby incorporated by reference.

### B. PCR Amplification

In some embodiments, Rac1b expression is detected using a polymerase chain reaction (PCR) assay to detect Rac1b expression.

### 1. Primers

Primers can be designed using the sequences of SEQ ID NOS: 5-8 or the Rac1b genomic sequence and used assays to amplify and detect to detect Rac1b expression. In some embodiments, the amplified Rac1b sequence is detected by signal amplification in gel electrophoresis. The primers typically flank unique sequences that can be amplified by methods such as polymerase chain reaction (PCR) or reverse transcriptase PCR(RT-PCR). In yet another embodiment, elevated Rac1b expression is detected using an RT-PCR assay to detect Rac1b transcription levels.

Typically, the target primers are present in the amplification reaction mixture at a concentration of about 0. μM to about 1.0 μM, about 0.25 μM to about 0.9 μM, about 0.5 to about 0.75 μM, or about 0.6 μM. The primer length can be about 8 to about 100 nucleotides in length, about 10 to about 75 nucleotides in length, about 12 to about 50 nucleotides in length, about 15 to about 30 nucleotides in length, or about 19 nucleotides in length.

### 2. Buffer

Buffers that may be employed are borate, phosphate, carbonate, barbital, Tris, etc. based buffers. (See, U.S. Pat. No. 5,508,178). The pH of the reaction should be maintained in the range of about 4.5 to about 9.5. (See, U.S. Pat. No. 5,508,178. The standard buffer used in amplification reactions is a Tris based buffer between 10 and 50 mM with a pH of around 8.3 to 8.8. (See Innis et al., supra.). One of skill in the art will recognize that buffer conditions should be designed to allow for the function of all reactions of interest. Thus, buffer conditions can be designed to support the amplification reaction as well as any subsequent restriction enzyme reactions. A particular reaction buffer can be tested for its ability to support various reactions by testing the reactions both individually and in combination.

### 3. Salt Concentration

The concentration of salt present in the reaction can affect the ability of primers to anneal to the target nucleic acid. (See, Innis et al.). Potassium chloride can be added up to a concentration of about 50 mM to the reaction mixture to promote primer annealing. Sodium chloride can also be added to promote primer annealing. (See, Innis et al.).

### 4. Magnesium Ion Concentration

The concentration of magnesium ion in the reaction can affect amplification of the target sequence(s). (See, Innis et al.). Primer annealing, strand denaturation, amplification specificity, primer-dimer formation, and enzyme activity are all examples of parameters that are affected by magnesium concentration. (See, Innis et al.). Amplification reactions should contain about a 0.5 to 2.5 mM magnesium concentration excess over the concentration of dNTPs. The presence of magnesium chelators in the reaction can affect the optimal magnesium concentration. A series of amplification reactions can be carried out over a range of magnesium concentrations to determine the optimal magnesium concentration. The optimal magnesium concentration can vary depending on the nature of the target nucleic acid(s) and the primers being used, among other parameters.

### 5. Deoxynucleotide Triphosphate Concentration

Deoxynucleotide triphosphates (dNTPs) are added to the reaction to a final concentration of about 20μM to about 300 μM Typically, each of the four dNTPs (G, A, C, T) are present at equivalent concentrations. (See, Innis et al.).

### 6. Nucleic Acid Polymerase

A variety of DNA dependent polymerases are commercially available that will function using the methods and compositions of the present invention. For example, Taq DNA Polymerase may be used to amplify target DNA sequences. The PCR assay may be carried out using as an enzyme component a source of thermostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g. Thermus and Pyrococcus species. Concentration ranges of the polymerase may range from 1-5 units per reaction mixture. The reaction mixture is typically between 15 and 100 μl.

### 7. Other Agents

Additional agents are sometime added to the reaction to achieve the desired results. For example, DMSO can be added to the reaction, but is reported to inhibit the activity of Taq DNA Polymerase. Nevertheless, DMSO has been recommended for the amplification of multiple target sequences in the same reaction. (See, Innis et al. supra). Stabilizing agents such as gelatin, bovine serum albumin, and non-ionic detergents (e.g. Tween-20) are commonly added to amplification reactions. (See, Innis et al. supra).

### 8. Amplification

Amplification of an RNA or DNA template using reactions is well known (see, U.S. Pat. Nos. 4,683,195 and 4,683,202; PCR PROTOCOLS: A GUIDE TO METHODS AND APPLICATIONS (Innis et al., eds, 1990)). Methods such as polymerase chain reaction (PCR) and ligase chain reaction (LCR) can be used to amplify nucleic acid sequences of target DNA sequences directly from animal feed and animal feed components. The reaction is preferably carried out in a thermal cycler to facilitate incubation times at desired temperatures. Degenerate oligonucleotides can be designed to amplify target DNA sequence homologs using the known sequences that encode the target DNA sequence. Restriction endonuclease sites can be incorporated into the primers.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

### 9. Detection of Amplified Products

Any method known in the art can be used to detect the amplified products, including, for example solid phase assays, anion exchange high-performance liquid chromatography, and fluorescence labeling of amplified nucleic acids (see MOLECULAR CLONING: A LABORATORY MANUAL (Sambrook et al. eds. 3d ed. 2001); Reischl and Kochanowski, Mol. Biotechnol. 3 (1): 55-71 (1995)). Gel electrophoresis of the amplified product followed by standard analyses known in the art can also be used to detect and quantify the amplified product. Suitable gel electrophoresis-based techniques include, for example, gel electrophoresis followed by quantification of the amplified product on a fluorescent automated DNA sequencer (see, e.g., Porcher et al., Biotechniques 13 (1): 106-14 (1992)); fluorometry (see, e.g., Innis et al., supra), computer analysis of images of gels stained in intercalating dyes (see, e.g., Schneeberger et al., PCR Methods Appl. 4 (4): 234-8 (1995)); and measurement of radioactivity incorporated during amplification (see, e.g., Innis et al., supra). Other suitable methods for detecting amplified products include using dual labeled probes, e.g., probes labeled with both a reporter and a quencher dye, which fluoresce only when bound to their target sequences; and using fluorescence resonance energy transfer (FRET) technology in which probes labeled with either a donor or acceptor label bind within the amplified fragment adjacent to each other, fluorescing only when both probes are bound to their target sequences. Suitable reporters and quenchers include, for example, black hole quencher dyes (BHQ), TAMRA, FAM, CY3, CY5, Fluorescein, HEX, JOE, LightCycler Red, Oregon Green, Rhodamine, Rhodamine Green, Rhodamine Red, ROX, TAMRA, TET, Texas Red, and Molecular Beacons.

The amplification and detection steps can be carried out sequentially, or simultaneously. In some embodiments, RealTime PCR is used to detect target sequences. For example, Real-time PCR using SYBR™ Green I can be used to amplify and detect the target nucleic acids (see, e.g., Ponchel et al., BMC Biotechnol. 3:18 (2003)). SYBR™ Green I only fluoresces when bound to double-stranded DNA (dsDNA). Thus, the intensity of the fluorescence signal depends on the amount of dsDNA that is present in the amplified product. Specificity of the detection can conveniently be confirmed using melting curve analysis.

### C. Immunoassays

In another embodiment, elevated Rac1b expression is detected using an immunochemical assay to detect Rac1b protein levels. Anti-Rac1b specific antibodies can be made by general methods known in the art. A preferred method of generating these antibodies is by first synthesizing peptide fragments. These peptide fragments should likely cover unique coding regions in the candidate gene. Since synthesized peptides are not always immunogenic by their own, the peptides should be conjugated to a carrier protein before use. Appropriate carrier proteins include but are not limited to Keyhole limpet hemacyanin (KLH). The conjugated phosphopeptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal (e.g., anti-Rac1b) antibodies can be purified by passing the harvested antibodies through an affinity column. Monoclonal antibodies are preferred over polyclonal antibodies and can be generated according to standard methods known in the art of creating an immortal cell line which expresses the antibody.

Nonhuman antibodies are highly immunogenic in human and that limits their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. and disclosed by van de Winkel, in U.S. Pat. No. 6,111,166 and hereby incorporated by reference in its entirety. “HuMAb-Mouse” is a strain of transgenic mice which harbor the entire human immunoglobin (Ig) loci and thus can be used to produce fully human monoclonal antibodies such as monoclonal anti-Rac1b antibodies.

Rac1b polypeptides and antibodies that specifically bind to them can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case Rac1b or an immunogenic fragment thereof). The antibody (e.g., anti-Rac1b) may be produced by any of a number of means well known to those of skill in the art and as described above. Alternatively, a protein or antigen of choice (in this case Rac1b, or an immunogenic fragment thereof) may be used to bind antibodies that specifically bind to the protein or antigen. The protein or antigen may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled Rac1b polypeptide or a labeled anti-Rac1b antibody. Alternatively, the labeling agent may be a third moiety, such a secondary antibody, which specifically binds to the antibody/Rac1b complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111: 1401-1406 (1973); Akerstrom et al., J. Immunol. 135:2589-2542 (1985)). The labeling agent can be modified with a detectable moiety, such as biotin, to which another molecule can specifically bind, such as streptavidin. The streptavidin may be bound to a label or detectable group as discussed below. A variety of detectable moieties are well known to those skilled in the art.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or 32P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize Rac1b, or secondary antibodies that recognize anti-Rac1b antibodies.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazined-iones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Western blot (immunoblot) analysis can also be used to detect and quantify the presence of the Rac1b polypeptides in a sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind Rac1b polypeptides. The anti-Rac1b antibodies specifically bind to Rac1b on the solid support, thereby forming an antibody-polypeptide complex. These antibodies may be directly labeled or alternatively may be subsequently detected using labeled antibodies (e.g., labeled sheep anti-mouse antibodies) that specifically bind to the anti-Rac1b antibodies.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev. 5:34-41 (1986)).

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

### IV. Modulation of Rac1b

In another embodiment, the invention provides methods for inhibiting MMP (e.g., MMP-3 or MMP-9) induced malignant transformation by modulating (e.g., inhibiting or enhancing) expression and/or function of Rac1b. For example, Rac1b expression can be inhibited using siRNA molecules and Rac1b function can be inhibited using antibodies that specifically bind to Rac1b. Strong Pearson correlations between target gene amplification/expression levels and pro-apoptotic effects of siRNAs will indicate that copy number/expression levels determine the extent of apoptotic responses to target gene inhibitors. Spearman rank test correlations between amplification detected and the level of induced apoptosis will indicate that the FISH test predicts response to targeted therapeutics.

In one embodiment, Rac1b expression will be downregulated using compounds that selectively kill cells that overexpress Rac1b. It is contemplated that such down regulation will decrease ROS production, this preventing tumor formation and decreasing the rate of malignant transformation.

In a preferred embodiment, identifying genes that are overexpressed in regions of amplification associated with reduced survival duration and for which inhibitors induce apoptosis in ovarian cancer cell lines in which the target is amplified is performed as described in Example 1 using RAC1B as the prototype. However, levels of amplification and gene expression may vary substantially between serous ovarian cancers. These quantitative differences and the presence of other aberrations may influence the degree of response to amplicon gene targeted therapies.

The invention further provides for compounds to treat a subject with elevated Rac1b expression. In a preferred embodiment, the compound is a Rac1b inhibitor such as, an antisense oligonucleotide; a siRNA olignonucleotide; a small molecule that interferes with Rac1b function; a viral vector producing a nucleic acid sequence that inhibits Rac1b; or an aptamer.

High throughput methods can be used to identify Rac1b inhibitors such as siRNA and/or small molecular inhibitor formulations to deliver Rac1b inhibitors efficiently to cultured cells and xenografts. Rac1b inhibitory formulations will be preferentially effective against xenografts that are amplified at the target loci and that these formulations will inhibit the formation or development of cancer. Effective formulations using such methods as described herein will be developed for clinical application.

### V. Compositions And Methods For Modulating Rac1b

In one embodiment, the invention provides compositions and methods for modulating (i.e., inhibiting or enhancing) Rac1b. Compounds (including, e.g., oligonucleotides) that inhibit Rac1b expression can be identified using methods known in the art. Oligonucleotide sequences that inhibit Rac1b include, but are not limited to, siRNA oligonucleotides, antisense oligonucleotides, peptide inhibitors and aptamer sequences that bind and act to inhibit RAC1B expression and/or function.

### A. RNA Interference

In one embodiment, RNA interference is used to generate small double-stranded RNA (small interference RNA or siRNA) inhibitors to affect the expression of Rac1b generally through cleaving and destroying its cognate RNA. Small interference RNA (siRNA) is typically 19-22 nt double-stranded RNA. siRNA can be obtained by chemical synthesis or by DNA-vector based RNAi technology. Using DNA vector based siRNA technology, a small DNA insert (about 70 bp) encoding a short hairpin RNA targeting the gene of interest is cloned into a commercially available vector. The insert-containing vector can be transfected into the cell, and expressing the short hairpin RNA. The hairpin RNA is rapidly processed by the cellular machinery into 19-22 nt double stranded RNA (siRNA). In a preferred embodiment, the siRNA is inserted into a suitable RNAi vector because siRNA made synthetically tends to be less stable and not as effective in transfection.

siRNA can be made using methods and algorithms such as those described by Wang L, Mu FY. (2004) A Web-based Design Center for Vector-based siRNA and siRNA cassette. Bioinformatics. (In press); Khvorova A, Reynolds A, Jayasena S D. (2003) Functional siRNAs and miRNAs exhibit strand bias. Cell. 115 (2):209-16; Harborth et al. (2003) Antisense Nucleic Acid Drug Dev. 13 (2): 83-105; Reynolds et al. (2004) Nat Biotechnol. 22 (3):326-30 and Ui-Tei et al., (2004) Nucleic Acids Res. 32 (3):936-48, which are hereby incorporated by reference.

Other tools for constructing siRNA sequences are web tools such as the siRNA Target Finder and Construct Builder available from GenScript (http://www.genscript.com), Oligo Design and Analysis Tools from Integrated DNA Technologies (http://www.idtdna.com/SciTools/SciTools.aspx), or siDESIGN™ Center from Dharmacon, Inc. (URL:=<http://design.dharmacon.com/default.aspx?source=0>). siRNA are suggested to be built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, since the function of siRNAs is to cleave mRNA sequences, it is important to use the mRNA nucleotide sequence and not the genomic sequence for siRNA design, although as noted in the Examples, the genomic sequence can be successfully used for siRNA design. However, designs using genomic information might inadvertently target introns and as a result the siRNA would not be functional for silencing the corresponding mRNA.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off-target effects is to screen the siRNA for target specificity.

In one embodiment, the siRNA can be modified on the 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

In a preferred embodiment, SEQ ID NO: 5 is used to design siRNA targeting Rac1b. For example, the four siRNAs comprising the sequences set forth in SEQ ID NOS: 1-4 were designed using design methods and algorithms known in the art (see, e.g., Reynolds et al., Nat Biotechnol. 22 (3):326-30 (2004). Factors used in designing the siRNA include, e.g., low G/C content, a bias towards low internal stability at the sense strand 3′-terminus, lack of inverted repeats, and sense strand base preferences (e.g., positions 3, 10, 13 and 19).

In another embodiment, web-based siRNA designing tools from Genescript (URL:=<http://www.genscript.com/rnai.html#design>) may be used to design siRNA sequences that target Rac1b since. Such tools typically provide the top candidate siRNA sequence and also perform BLAST screening (Altschul et al. (1990) “Basic local alignment search tool.” J. Mol. Biol. 215:403-410) on each resulting siRNA sequence.

### B. Antisense Oligonucleotides

In another embodiment, antisense oligonucleotides which inhibit RAC1b and other candidate genes can be designed. Antisense oligonucleotides are short single-stranded nucleic acids, which function by selectively hybridizing to their target mRNA, thereby blocking translation. Translation is inhibited by either RNase H nuclease activity at the DNA:RNA duplex, or by inhibiting ribosome progression, thereby inhibiting protein synthesis. This results in discontinued synthesis and subsequent loss of function of the protein for which the target mRNA encodes.

In a preferred embodiment, antisense oligos are phosphorothioated upon synthesis and purification, and are usually 18-22 bases in length. It is contemplated that the Rac1b and other candidate gene antisense oligos may have other modifications such as 2′-O-Methyl RNA, methylphosphonates, chimeric oligos, modified bases and many others modifications, including fluorescent oligos.

In a preferred embodiment, active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA (e.g., Rac1b mRNA SEQ ID NO: 8) will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20° C. and stable for several weeks.

### C. Aptamers

In another embodiment, aptamer sequences which bind to specific RNA or DNA sequences can be made. Aptamer sequences can be isolated through methods such as those disclosed in co-pending U.S. patent application Ser. No. 10/934,856 (published as U.S. Patent Publication No. 20050142582), which is hereby incorporated by reference.

It is contemplated that the sequences described herein may be varied to result in substantially homologous sequences which retain the same function as the original. As used herein, a polynucleotide or fragment thereof is “substantially homologous” (or “substantially similar”) to another if, when optimally aligned (with appropriate nucleotide insertions or deletions) with the other polynucleotide (or its complementary strand), using an alignment program such as BLASTN (Altschul et al. (1990) J. Mol. Biol. 215:403-410), and there is nucleotide sequence identity in at least about 80%, preferably at least about 90%, and more preferably at least about 95-98% of the nucleotide bases.

### D. Recombinant Expression of Rac1b Modulators

Rac1b modulators such as the siRNA Rac1b inhibitors described herein can also be expressed recombinantly. In general, the nucleic acid sequences encoding Rac1b inhibitors such as the siRNA Rac1b inhibitor and related nucleic acid sequence homologues can be cloned. This aspect of the invention relies on routine techniques in the field of recombinant genetics. Generally, the nomenclature and the laboratory procedures in recombinant DNA technology described herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 1994)).

Nucleic acids encoding sequences of Rac1b modulators can also be isolated from expression libraries using antibodies as probes. Such polyclonal or monoclonal antibodies can be raised using, for example, the polypeptides comprising the sequences set forth in SEQ ID NOS: 6 and 9 and subsequences thereof, or polypeptides encoded by the sequences set forth in SEQ ID NOS: 5 and 8, and subsequences thereof, using methods known in the art (see, e.g., Harlow and Lane, Antibodies: A Laboratory Manual (1988)).

### E. Antibodies That Specifically Bind Rac1b

In some embodiments, the Rac1B modulator is an antibody (e.g., a polyclonal or monoclonal antibody) that specifically binds and/or inhibits Rac1b which can be used using methods known in the art and may be used therapeutically as well. Such use of antibodies has been demonstrated by others and may be useful in the present invention to inhibit or downregulate Rac1b. Rac1b specific antibodies can be made by a number of methods known in the art. In one embodiment, specific Rac1b antibodies are generated by first amplifying and cloning cDNA fragments of SEQ ID NOS: 5 or 8. A sequence such as SEQ ID NO: 5 is amplified and cloned, and then expressed peptide fragments of Rac1b from the cloned cDNAs are obtained. In another embodiment, peptide fragments are synthesized to generate peptide fragments such as SEQ ID NOS: 6 and 9. These peptide fragments should include portions of the Rac1b isoform insertion and may contain the adjacent Rac1 amino acid sequence. It is preferred that no more than 14 amino acids of the wild-type Rac1 protein sequence are used in conjunction with portions of the Rac1b 19 amino acid insertion, so as to generate very specific Rac1b antibodies. For example, the Rac1b antibody described herein was raised against the synthesized peptide Ac-CGKDRPSRGKDKPIA-amide (SEQ ID NO: 9), which is a portion of the Rac1b amino acid insertion shown in SEQ ID NO: 6.

Since synthesized peptides are not always immunogenic on their own, the peptides are conjugated to a carrier protein before use. Appropriate carrier proteins include, but are not limited to, Keyhole limpet hemacyanin (KLH), bovine serum albumin (BSA) and ovalbumin (OVA). The conjugated peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal Rac1b antibodies can be purified by passing the harvested antibodies through an affinity column. However, monoclonal antibodies are preferred over polyclonal antibodies and can be generated according to standard methods known in the art of creating an immortal cell line which expresses the antibody.

Nonhuman antibodies are highly immunogenic in human thus limiting their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. (Princeton, N.J.). “HuMAb-Mouse” is a strain of transgenic mice that harbors the entire human immunoglobin (Ig) loci and thus can be used to produce fully human monoclonal Rac1b antibodies.

Immunoblotting using the specific antibodies of the invention with Rac1 sequence should not produce a detectable signal at preferably 0.5-10 fold molar excess (relative to the Rac1b detection), more preferably at 50 fold molar excess and most preferably no signal is detected at even 100 fold molar excess.

Substantially identical nucleic acids encoding sequences of Rac1b inhibitors can be isolated using nucleic acid probes and oligonucleotides under stringent hybridization conditions, by screening libraries. Alternatively, expression libraries can be used to clone these sequences, by detecting expressed homologues immunologically with antisera or purified antibodies made against the core domain of nucleic acids encoding Rac1b inhibitor sequences.

Gene expression of RAC1B can also be analyzed by techniques known in the art, e.g., reverse transcription and amplification of mRNA, isolation of total RNA or poly A+RNA, northern blotting, dot blotting, in situ hybridization, RNase protection, probing DNA microchip arrays, and the like.

To obtain high level expression of a cloned gene or nucleic acid sequence, such as those cDNAs encoding nucleic acid sequences encoding Rac1b, Rac1b inhibitors such as the siRNA Rac1b inhibitor and related nucleic acid sequence homologues, one typically subclones a sequence (e.g., nucleic acid sequences encoding Rac1b and Rac1b inhibitors such as the siRNA Rac1b inhibitor and related nucleic acid sequence homologue or a sequence encoding SEQ ID NOS: 1-4) into an expression vector that is subsequently transfected into a suitable host cell. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding Rac1b inhibitors such as the siRNA Rac1b inhibitor or a subsequence thereof. Suitable bacterial promoters are well known in the art and described, e.g., in Sambrook et al. and Ausubel et al. The elements that are typically included in expression vectors also include a replicon that functions in a suitable host cell such as E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to the recombinant RAC1B inhibitors peptides to provide convenient methods of isolation, e.g., His tags. In some case, enzymatic cleavage sequences (e.g., SEQ ID NO: 10, Met-(His)g-Ile-Glu-Gly-Arg which form the Factor Xa cleavage site) are added to the recombinant Rac1b inhibitor peptides. Bacterial expression systems for expressing the Rac1b inhibitor peptides and nucleic acids are available in, e.g. E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Standard transfection methods are used to produce cell lines that express large quantities of Rac1b inhibitor, which can then purified using standard techniques (see, e.g. Colley et al., J. Biol. Chem. 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact. 132:349-351 (1977); Clark-Curtiss & Curtiss, Methods in Enzymology 101:347-362 (Wu et al., eds, 1983). For example, any of the well known procedures for introducing foreign nucleotide sequences into host cells may be used. These include the use of calcium phosphate transfection, lipofectamine, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing RAC1B inhibitor peptides and nucleic acids.

After the expression vector is introduced into the cells, the transfected cells are cultured under conditions favoring expression of Rac1b inhibitors such as the siRNA Rac1b inhibitor and related nucleic acid sequence homologues.

### VI. Methods of Treatment

In some embodiments, the invention provides methods of treating disorders associated with overexpression of Rac1b, i.e., MMP (e.g., MMP-3 and MMP-9) induced malignancies. The Rac1b modulator antibodies, peptides and nucleic acids of the present invention, such as the siRNA that specifically targets Rac1b, also can be used to treat or prevent a variety of disorders associated with MMP (e.g., MMP-3 OR MMP-9) induced cancer. The antibodies, peptides and nucleic acids are administered to a patient in an amount sufficient to elicit a therapeutic response in the patient (e.g., inhibiting the development, growth or metastasis of cancerous cells; reduction of tumor size and growth rate, prolonged survival rate, reduction in concurrent cancer therapeutics administered to patient). An amount adequate to accomplish this is defined as “therapeutically effective dose or amount.”

The antibodies, peptides and nucleic acids of the invention can be administered directly to a mammalian subject using any route known in the art, including e.g., by injection (e.g., intravenous, intraperitoneal, subcutaneous, intramuscular, or intradermal), inhalation, transdermal application, rectal administration, or oral administration.

In other embodiments, such antibodies that specifically bind or inhibit Rac1b, may be used therapeutically. Such use of antibodies has been demonstrated by others and may be useful in the present invention to inhibit or downregulate Rac1b.

### VII. High Throughput Screening For Small Molecules That Modulate Rac1b

In one embodiment, high throughput screening (HTS) methods are used to identify compounds that modulate Rac1b, e.g., inhibit or enhance Rac1b expression. HTS methods involve providing a combinatorial chemical or peptide library containing a large number of potential therapeutic compounds (i.e., compounds that inhibit Rac1b). Such “libraries” are then screened in one or more assays, as described herein, to identify those library members (particular peptides, chemical species or subclasses) that display the desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14 (3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., ECIS TM, Applied BioPhysics Inc., Troy, N.Y., MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g. ComGenex, Princeton, N.J., Tripos, Inc., St. Louis, Mo., 3D Pharmaceuticals, Exton, Pa., Martek Biosciences, Columbia, Md., etc.).

### A. Pharmaceutical Compositions

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

### B. Gene Therapy

In certain embodiments, the nucleic acids encoding inhibitory Rac1b peptides and nucleic acids of the present invention can be used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses an inhibitory RAC1 B peptides and nucleic acids of the present invention, thereby mitigating the effects of over amplification of a candidate gene associated with reduced survival rate.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6 (10): 1149-1154 (1998); Vigne, Restorative Neurology and Neuroscience 8:35-36 (1995); Kremer & Perricaudet, British Medical Bulletin 51 (1):31-44 (1995); Haddada et al., in Current Topics in Microbiology and Immunology (Doerfler & Böhm eds., 1995); and Yu et al., Gene Therapy 1: 13-26 (1994)).

For delivery of nucleic acids, viral vectors may be used. Suitable vectors include, for example, herpes simplex virus vectors as described in Lilley et al., Curr. Gene Ther. 1 (4):339-58 (2001), alphavirus DNA and particle replicons as described in e.g., Polo et al., Dev. Biol. (Basel) 104:181-5 (2000), Epstein-Barr virus (EBV)-based plasmid vectors as described in, e.g., Mazda, Curr. Gene Ther. 2 (3):379-92 (2002), EBV replicon vector systems as described in e.g., Otomo et al., J. Gene Med. 3 (4):345-52 (2001), adeno-virus associated viruses from rhesus monkeys as described in e.g., Gao et al., PNAS USA. 99 (18):11854 (2002), adenoviral and adeno-associated viral vectors as described in, e.g., Nicklin and Baker, Curr. Gene Ther. 2 (3):273-93 (2002). Other suitable adeno-associated virus (AAV) vector systems can be readily constructed using techniques well known in the art (see, e.g. U.S. Pat. Nos. 5,173,414 and 5,139,941; PCT Publication Nos. WO 92/01070 and WO 93/03769; Lebkowski et al. (1988) Mol. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling and Smith (1994) Gene Therapy 1:165-169; and Zhou et al. (1994) J. Exp. Med. 179:1867-1875). Additional suitable vectors include E1B gene-attenuated replicating adenoviruses described in, e.g. Kim et al., Cancer Gene Ther.9 (9):725-36 (2002) and nonreplicating adenovirus vectors described in e.g., Pascual et al., J. Immunol. 160 (9):4465-72 (1998). Exemplary vectors can be constructed as disclosed by Okayama et al. (1983) Mol. Cell. Biol. 3:280.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery according to the methods of the invention.

In one illustrative embodiment, retroviruses provide a convenient and effective platform for gene delivery systems. A selected nucleotide sequence encoding an inhibitory RAC1 B nucleic acid or polypeptide can be inserted into a vector and packaged in retroviral particles using techniques known in the art. The recombinant virus can then be isolated and delivered to a subject. Suitable vectors include lentiviral vectors as described in e.g., Scherr and Eder, Curr. Gene Ther. 2 (1):45-55 (2002). Additional illustrative retroviral systems have been described (e.g., U.S. Pat. No. 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Curr. Opin. Genet. Develop. 3:102-109.

Other known viral-based delivery systems are described in, e.g., Fisher-Hoch et al. (1989) Proc. Natl. Acad. Sci. USA 86:317-321; Flexner et al. (1989) Ann. N.Y. Acad. Sci. 569:86-103; Flexner et al. (1990) Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502; Guzman et al. (1993) Circulation 88:2838-2848; Guzman et al. (1993) Cir. Res. 73:1202-1207; and Lotze and Kost, Cancer Gene Ther. 9 (8):692-9 (2002).

### C. Combination Therapy

In some embodiments, the Rac1b modulator (e.g., inhibitory Rac1b polypeptides and nucleic acids) are administered in combination with a second therapeutic agent for treating or preventing cancer. In one embodiment, an inhibitory Rac1b siRNA may be administered in conjunction with a second therapeutic agent for treating or preventing breast, ovarian or colon cancer. For example, an inhibitory Rac1b siRNA of SEQ ID NO: 3 and 4 may be administered in conjunction with any of the standard treatments for ovarian cancer including, but not limited to, chemotherapeutic agents including, e.g., alitretinoin, altretamine, anastrozole, azathioprine, bicalutamide, busulfan, capecitabine, carboplatin, cisplatin, cyclophosphamide, cytarabine, doxorubicin, epirubicin, etoposide, exemestane, finasteride, fluorouracil, fulvestrant, gemtuzumab, ozogamicin, hydroxyurea, ibritumomab, idarubicin, ifosfamide, imatinib, letrozole, megestrol acetate, methotrexate, mifepristone, paclitaxel, rituximab, tamoxifen, temozolomide, tretinoin, triptorelin, vincristine, or vinorelbine, and radiation treatment.

The Rac1b modulator (e.g., inhibitory Rac1b polypeptides and nucleic acids) and the second therapeutic agent may be administered simultaneously or sequentially. For example, the inhibitory Rac1b polypeptides and nucleic acids may be administered first, followed by the second therapeutic agent. Alternatively, the second therapeutic agent may be administered first, followed by the inhibitory Rac1b polypeptides and nucleic acids. In some cases, the inhibitory Rac1b polypeptides and nucleic acids and the second therapeutic agent are administered in the same formulation. In other cases the inhibitory Rac1b polypeptides and nucleic acids and the second therapeutic agent are administered in different formulations. When the inhibitory Rac1b polypeptides and nucleic acids and the second therapeutic agent are administered in different formulations, their administration may be simultaneous or sequential.

In some cases, the inhibitory Rac1b polypeptides and nucleic acids can be used to target therapeutic agents to cells and tissues expressing Rac1b and other candidate genes that are related to reduced survival rate.

### D. Administration

Administration of the Rac1b modulators (e.g., antibodies, peptides and nucleic acids) of the invention can be in any convenient manner, e.g., by injection, intratumoral injection, intravenous and arterial stents (including eluting stents), cather, oral administration, inhalation, transdermal application, or rectal administration. In some cases, the peptides and nucleic acids are formulated with a pharmaceutically acceptable carrier prior to administration. Pharmaceutically acceptable carriers are determined in part by the particular composition being administered (e.g., nucleic acid or polypeptide), as well as by the particular method used to administer the composition. Accordingly, there are a wide variety of suitable formulations of pharmaceutical compositions of the present invention (see, e.g. Remington's Pharmaceutical Sciences, 17^{th} ed., 1989).

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector to be administered in the treatment or prophylaxis of diseases or disorder associated with the disease, the physician evaluates circulating plasma levels of the polypeptide or nucleic acid, polypeptide or nucleic acid toxicities, progression of the disease (e.g., ovarian cancer), and the production of antibodies that specifically bind to the peptide. Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked c acid is from about 1 μg to about 100 μg for a typical 70 kilogram patient, and doses of vectors which include a viral particle are calculated to yield an equivalent amount of therapeutic nucleic acid.

For administration, Rac1b modulators (e.g., antibodies, polypeptides and nucleic acids) of the present invention can be administered at a rate determined by the LD-50 of the polypeptide or nucleic acid, and the side-effects of the antibody, polypeptide or nucleic acid at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

In certain circumstances it will be desirable to deliver the pharmaceutical compositions comprising the Rac1b modulators (e.g., antibodies, peptides and nucleic acids) of the present invention parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

To date, most siRNA studies have been performed with siRNA formulated in sterile saline or phosphate buffered saline (PBS) that has ionic character similar to serum. There are minor differences in PBS compositions (with or without calcium, magnesium, etc.) and investigators should select a formulation best suited to the injection route and animal employed for the study. Lyophilized oligonucleotides and standard or stable siRNAs are readily soluble in aqueous solution and can be resuspended at concentrations as high as 2.0 mM. However, viscosity of the resultant solutions can sometimes affect the handling of such concentrated solutions.

While lipid formulations have been used extensively for cell culture experiments, the attributes for optimal uptake in cell culture do not match those useful in animals. The principle issue is that the cationic nature of the lipids used in cell culture leads to aggregation when used in animals and results in serum clearance and lung accumulation. Polyethylene glycol complexed-liposome formulations are currently under investigation for delivery of siRNA by several academic and industrial investigators, including Dharmacon, but typically require complex formulation knowledge. There are a few reports that cite success using lipid-mediated delivery of plasmids or oligonucleotides in animals.

Oligonucleotides can also be administered via bolus or continuous administration using an ALZET mini-pump (DURECT Corporation). Caution should be observed with bolus administration as studies of antisense oligonucleotides demonstrated certain dosing-related toxicities including hind limb paralysis and death when the molecules were given at high doses and rates of bolus administration. Studies with antisense and ribozymes have shown that the molecules distribute in a related manner whether the dosing is through intravenous (IV), subcutaneous (sub-Q), or intraperitoneal (IP) administration. For most published studies, dosing has been conducted by IV bolus administration through the tail vein. Less is known about the other methods of delivery, although they may be suitable for various studies. Any method of administration will require optimization to ensure optimal delivery and animal health.

For bolus injection, dosing can occur once or twice per day. The clearance of oligonucleotides appears to be biphasic and a fairly large amount of the initial dose is cleared from the urine in the first pass. Dosing should be conducted for a fairly long term, with a one to two week course of administration being preferred. This is somewhat dependent on the model being examined, but several metabolic disorder studies in rodents that have been conducted using antisense oligonucleotides have required this course of dosing to demonstrate clear target knockdown and anticipated outcomes.

In certain embodiments, the inventors contemplate the use of liposomes, nanocapsules, microparticles, microspheres, lipid particles, vesicles, and the like, for the administration of the Rac1b inhibitory peptides and nucleic acids of the present invention. In particular, the compositions of the present invention may be formulated for delivery either encapsulated in or operatively attached to a lipid particle, a liposome, a vesicle, a nanosphere, or a nanoparticle or the like.

The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 Å, containing an aqueous solution in the core.

Alternatively, the invention provides for pharmaceutically-acceptable nanocapsule formulations of the compositions of the present invention. Nanocapsules can generally entrap compounds in a stable and reproducible way (Henry-Michelland et al. Attachment of antibiotics to nanoparticles: preparation, drug-release and antimicrobial activity in vitro, Int. J. Pharm. 35, 121-27, 1987; Quintanar-Guerrero et al. Pseudolatex preparation using a novel emulsion-diffusion process involving direct displacement of partially water-miscible solvents by distillation. Int'l J. Pharmaceutics 188 (2), 155-64, 1998; Douglas et al. Nanoparticles in drug delivery. Rev. Ther. Drug Carrier Syst. 3, 233-61, 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use in the present invention. Such particles may be are easily made, as described (Couvreur et al., Tissue distribution of antitumor drugs associated with polyalkylcyanoacrylate nanoparticles. J. Pharm. Sci. 69, 199, 1980; zur Muhlen et al. Solid lipid nanoparticles (SLN) for controlled drug delivery—Drug release and release mechanism. Euro. J. Pharmaceutics and Biopharmaceutics 45 (2), 149-55, 1998; Zambaux et al. Influence of experimental parameters on characteristics of poly(lactic acid) nanoparticles prepared by a double emulsion method. J. Controlled Release 50 (1-3), 31-40, 1998; (H. Pinto-Alphandry, A. Andremont and P. Couvreur, Targeted delivery of antibiotics using liposomes and nanoparticles: research and applications. Int. J. Antimicrob. Agents 13, 155-168, 2000); U.S. Pat. No. 5,145,684; and U.S. Pat. No. 6,881,421).

### VIII. Kits

The present invention further provides kits for use within any of the above diagnostic methods. Such kits typically comprise two or more components necessary for performing a diagnostic assay. Components may be compounds, reagents, containers and/or equipment. For example, one container within a kit may contain an inhibitory Rac1b polypeptides and nucleic acids. One or more additional containers may enclose elements, such as reagents or buffers, to be used in the assay. Such kits may also, or alternatively, contain a detection reagent as described above that contains a reporter group suitable for direct or indirect detection of antibody binding.

Kits can also be supplied for therapeutic uses. Thus, the subject composition of the present invention may be provided, usually in a lyophilized form, in a container. The inhibitory Rac1b polypeptides and nucleic acids described herein are included in the kits with instructions for use, and optionally with buffers, stabilizers, biocides, and inert proteins. Generally, these optional materials will be present at less than about 5% by weight, based on the amount of polypeptide or nucleic acid, and will usually be present in a total amount of at least about 0.001% by weight, based on the polypeptide or nucleic acid concentration. It may be desirable to include an inert extender or excipient to dilute the active ingredients, where the excipient may be present in from about 1 to 99% weight of the total composition. The kits may further comprise a second therapeutic agent, e.g., paclitaxel, carboplatin, or other chemotherapeutic agent.

### EXAMPLES

The following examples are offered to illustrate, but not to limit the presently claimed invention.

### Example 1

### Methods

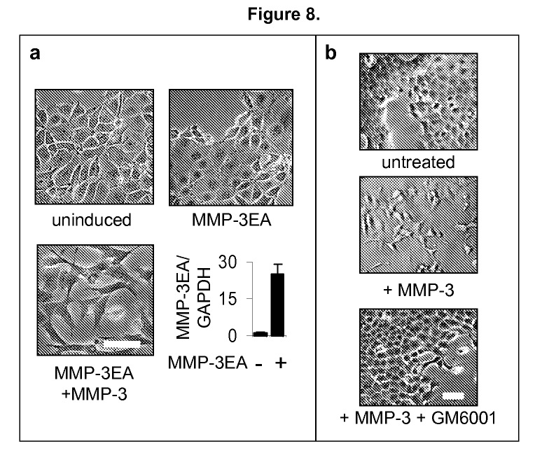

Cell culture, antibodies, and plasmids. Cell culture was as previously described (Lochter, A. et al. Misregulation of stromelysin-1 expression in mouse mammary tumor cells accompanies acquisition of stromelysin-1-dependent invasive properties. J. Biol Chem 272, 5007-15, 1997; Lochter, A. et al. Matrix metalloproteinase stromelysin-1 triggers a cascade of molecular alterations that leads to stable epithelial-to-mesenchymal conversion and a premalignant phenotype in mammary epithelial cells. J Cell Biol 139, 1861-72, 1997); for gene repression, a 5 mg ml⁻¹ stock solution of tetracycline in 100% ethanol was diluted 1:1000 into culture medium and changed daily. To stimulate cells with MMP-3, we used medium that had been conditioned by SCp2 cells containing the tet-regulated, autoactivated MMP-3-construct (Lochter, A. et al. J Biol Chem 272, 5007-15, 1997; Lochter, A. et al. J Cell Biol 139, 1861-72, 1997) with expression induced by growth in the absence of tetracycline; conditioned medium from cells repressed by treatment with tetracycline was used for controls. This conditioned medium was analyzed by zymography to verify that only MMP-3 was being expressed, and was shown be active through extracellular proteolysis (FIG. 8).

Except as otherwise indicated, cells were incubated in the presence of conditioned medium containing MMP-3 for 4 days and with 25 μM H₂O₂ for 7 days. NAC was used at a concentration of 10 mM. Antibodies against cytokeratin and vimentin were described previously (Lochter, A. et al. J Biol Chem 272, 5007-15, 1997; Lochter, A. et al. J Cell Biol 139, 1861-72, 1997).

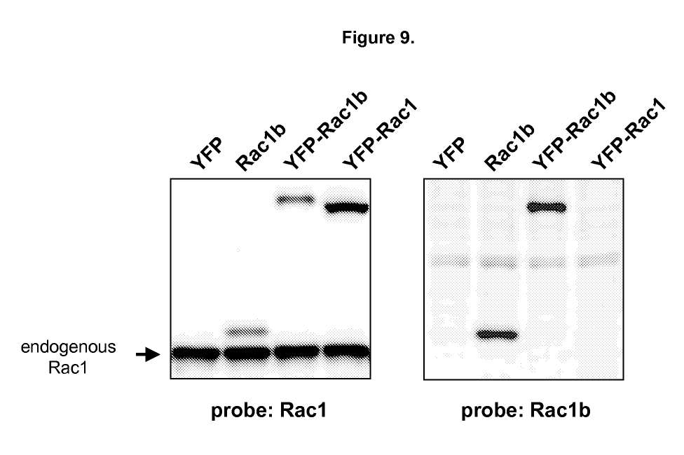

The Rac antibody was obtained from Upstate. The Rac1b antibody was raised against the peptide Ac-CGKDRPSRGKDKPIA-amide (SEQ ID NO: 9), using conventional antibody methods known in the art. FIG. 9 shows the validation of the Rac1b antibody. Cells were transfected with plasmids expressing YFP, cloned mouse Rac1b, YFP-Rac1b, or YFP-Rac1. Cell lysates were western blotted using anti-Rac1 antibody (1:1000, Upstate), or the rabbit antisera raised against the Rac1b insert peptide (1:100, Biosource). Note that Rac1 antibody cross-reacts with Rac1b, but that the Rac1b antibody does not recognize Rac1.

Human catalase cDNA was obtained from R. Arnold (Emory University, Atlanta, USA), human SOD1 and SOD2 cDNA were obtained from T.-T. Huang (Stanford University, Stanford, USA), SOD1, SOD2, and CAT were cloned into pcDNA3.1 expression vectors; all other constructs were subcloned into the tetracycline-repressible expression system used previously for expression of MMP-3 (described in Lochter, A. et al. Misregulation of stromelysin-1 expression in mouse mammary tumor cells accompanies acquisition of stromelysin-1-dependent invasive properties. J Biol Chem 272, 5007-15 (1997) and Lochter, A. et al. Matrix metalloproteinase stromelysin-1 triggers a cascade of molecular alterations that leads to stable epithelial-to-mesenchymal conversion and a premalignant phenotype in mammary epithelial cells. J Cell Biol 139, 1861-72 (1997)).

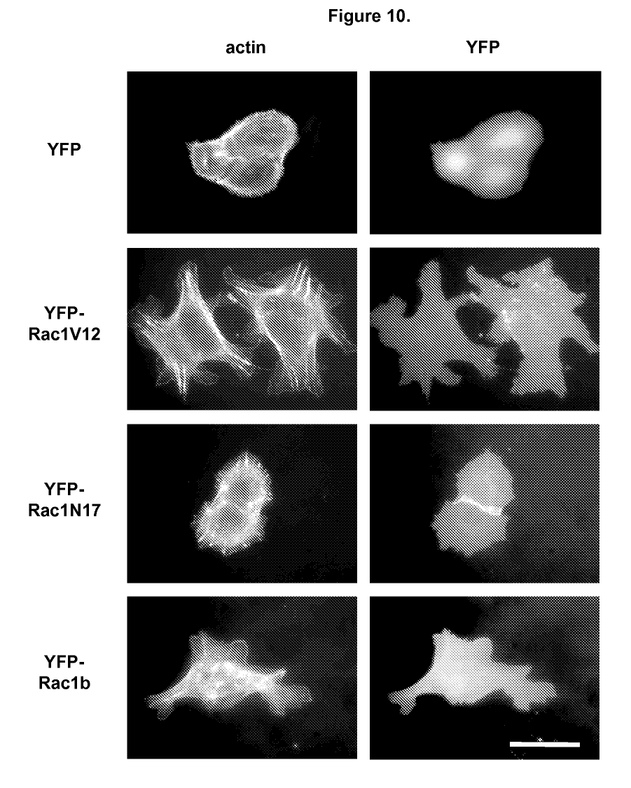

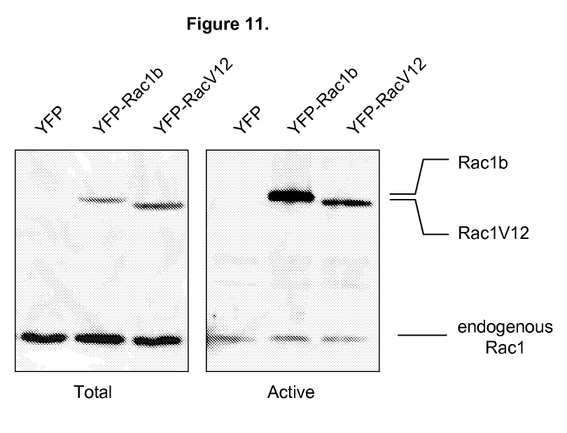

Rac1 and Rac1b were cloned from SCp2 cDNA and expressed as unmodified proteins or as fused with YFP. Rac1V12 and Rac11N17 mutants of Rac1 (FIGS. 10 and 11), as well as the catalytically inactive E217A mutant of MMP-3, were generated using the QUICKCHANGE mutagenesis kit (Stratagene); all modified plasmids were sequence-verified. Transcript levels were assessed using RT/PCR by isolating RNA (Tri-pure; Roche Diagnostics), synthesizing cDNA, and performing quantitative, real-time PCR (Lightcycler, Roche Diagnostics); all these experiments were normalized to GAPDH.

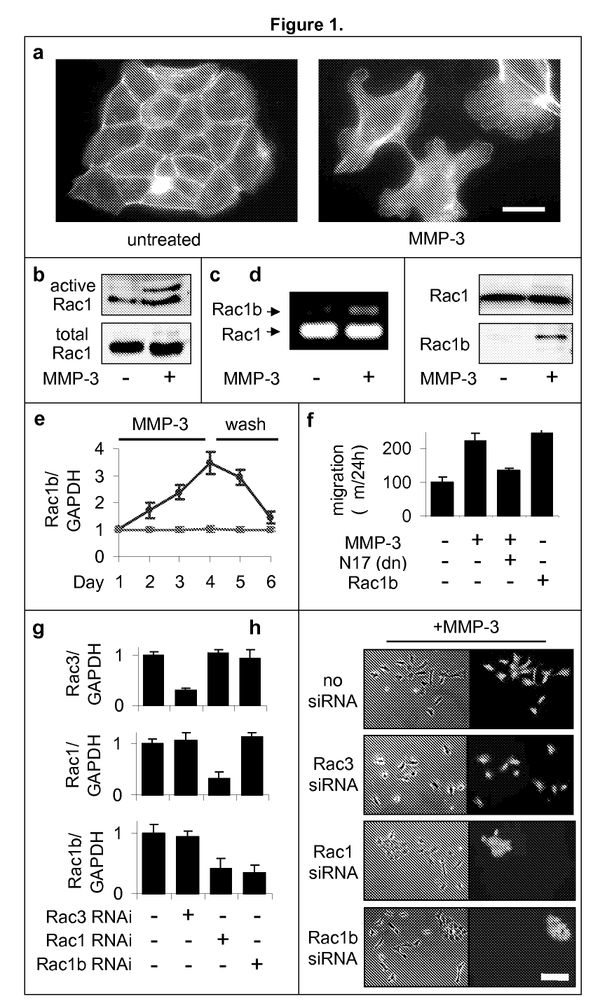

For analysis of Rac1 and Rac1b (FIG. 1c), oligonucleotide primers that hybridize to sequences flanking the splice insertion site were used; for specific analysis of Rac1b (FIG. 1d, 4h), oligonucleotide primers specific for the Rac1b splice isoform were used.

Rho GTPase assays. Cells were lysed in GST-Fish buffer (10% Glycerol, 50 mM Tris pH 7.4, 100 mM NaCl, 1% NP-40, 2 mM MgCl2, 10 μg ml-1 leupeptin, 10 μg ml-1 pepstatin, 10 μg ml-1 aprotinin, 10 μg ml-1 E 64, and 1 mM Pefabloc). Equal amounts of protein supernatants were incubated with GST-PAK-CD (Rac and Cdc42 binding domain) or GST-C21 (Rho binding domain) fusion protein-coated Sepharose beads on ice for 45 min. The beads were washed, eluted in sample buffer, and then analyzed by SDS-PAGE and Western blotting using antibodies against Rac, Cdc42, and Rho. Dominant negative and constitutively active Rac1 expression constructs were provided by D. Kalman (Emory University, Atlanta, USA). Rac1 and Rac3 siRNA were smartpool reagents (Dharmacon), while Rac1b siRNA used the sequence UGGAGACACAUGUGGUAAAGAUAGA (SEQ ID NO: 4); siRNA were transfected into SCp2 cells with Lipofectamine 2000 (Gibco) using the manufacturer's protocols. For analysis of endogenous gene knockdown, RNA was harvested after 24 hours and analyzed by RT/PCR using primer pairs selective for Rac1, Rac1b, or Rac3. For MMP-3-induced EMT, siRNA mixtures were cotransfected with YFP-C1 and then treated with MMP-3 for four days, and then evaluated for scatter of fluorescent (cotransfected with YFP and siRNA) and nonfluorescent (nontransfected control) colonies.

ROS and 8-oxoG analyses. To measure ROS concentrations, cells were incubated in the dark with 50 mM DCFDA (Molecular Probes) for 30 min in serum- and phenol-red-free medium. For 8-oxoG analysis, a modification of published techniques (Struthers, L., Patel, R., Clark, J. & Thomas, S. Direct detection of 8-oxodeoxyguanosine and 8-oxoguanine by avidin and its analogues. Anal Biochem 255, 20-31, 1998; Neumann, C. A. et al. Essential role for the peroxiredoxin Prdx1 in erythrocyte antioxidant defence and tumour suppression. Nature 424, 561-5, 2003) was used: cells were fixed in methanol (20 min, −20° C.), permeabilized with TBST (Tris-buffered saline+0.1% Triton-X100; 15 min, 25° C.), blocked for nonspecific binding (TBST+15% fetal calf serum; 2 hrs, 25° C.), and stained with 15 μg/ml. FITC-conjugated avidin (Sigma; 1 hr, 37° C.). To verify specificity of staining, FITC-avidin was preincubated with a 10-fold excess of either the blocking oligonucleotide 5′-GAA CTA GTN ATC CCC CGG GTC GC-3′ (where N is 8-oxodeoxyguanosine) (SEQ ID NO: 11), or the control oligonucleotide 5′-GAA CTA GTG ATC CCC CGG GTC GC-3′ (SEQ ID NO: 12). Images were captured using a Nikon Diaphot 300 microscope and Spot RT camera and software (Technical Instruments, Burlingame). Fluorescence was quantified using IMAGEJ (URL:=<http://rsb.info.nih.gov/ij/index.html>). For DCFDA staining, cellular fluorescence was quantified, for FITC-avidin staining, nuclear fluorescence was measured (using a DAPI image mask). More than 250 measurements were made for each data point. JC-1 and nitrobluetetrazolium labeling was performed essentially as in Werner, E. & Werb, Z. Integrins engage mitochondrial function for signal transduction by a mechanism dependent on Rho GTPases. J Cell Biol 158, 357-68 (2002).

Genomic instability assays. The PALA assay was performed essentially as previously described (Nieto, M. A. The snail superfamily of zinc-finger transcription factors. Nat Rev Mol Cell Biol 3, 155-66, 2002; Thiery, J. P. Epithelial-mesenchymal transitions in tumour progression. Nat Rev Cancer 2, 442-54, 2002). PALA, an inhibitor of the aspartate transcarbamylase activity of the multifunctional CAD enzyme, was obtained from the Drug Synthesis and Chemistry Branch, Developmental Therapeutics Program, Division of Cancer Treatment, National Cancer Institute, Bethesda, Md. SCp2 were exposed to MMP-3 or induced to express MMP-3, then MMP-3 was removed or repressed for 24-48 hours before cells were trypsinized, counted, and allowed to adhere to new dishes before being exposed to 200 μM PALA (the LC50 of PALA for SCp2 cells was determined to be 25 μM). Acquisition of resistance to PALA was assessed by counting the number of surviving colonies relative to the total cells plated. Unless otherwise indicated, PALA assays were performed on cells that had been treated for 14 days. For FISH analysis of the CAD locus, BAC probes (BACPAC, Oakland, Calif.) rp23-73H2O (mouse chromosome 9) and rp23-154k6 (mouse chromosome 5, containing CAD locus) were labeled using Bioprime DNA Labeling System (Invitrogen) using FITC-dUTP (NEN) for rp23-73H20 or Cy3-dUTP (Amersham) for rp23-154k6, and then hybridized to SCp2 cells that had been treated with MMP-3 or MMP-3 and PALA; the number of copies per cell was quantified using fluorescence microscopy. Genomic alterations were assayed using array-based comparative genomic hybridization (CGH) as previously described 30, except that mouse BAC arrays and mouse Cot-1 DNA (Invitrogen) were used instead of human Cot-1 DNA. Clonal populations were derived from SCp2 cells grown in the presence (clones N2, N11, N12, N13) or absence (clones D, F, I) of MMP-3 for 14 days, then selected for resistance to PALA in the absence of MMP-3. The reference DNA used for all CGH samples was derived from parental SCp2 cells (not treated with PALA or MMP-3), and all DNA was isolated using DNeasy Tissue Kit (Qiagen).

### Example 2

### MMP-3 Induces EMT Through Rac1b

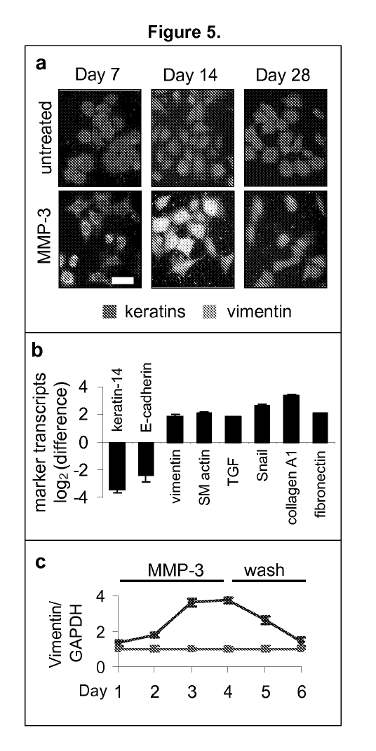

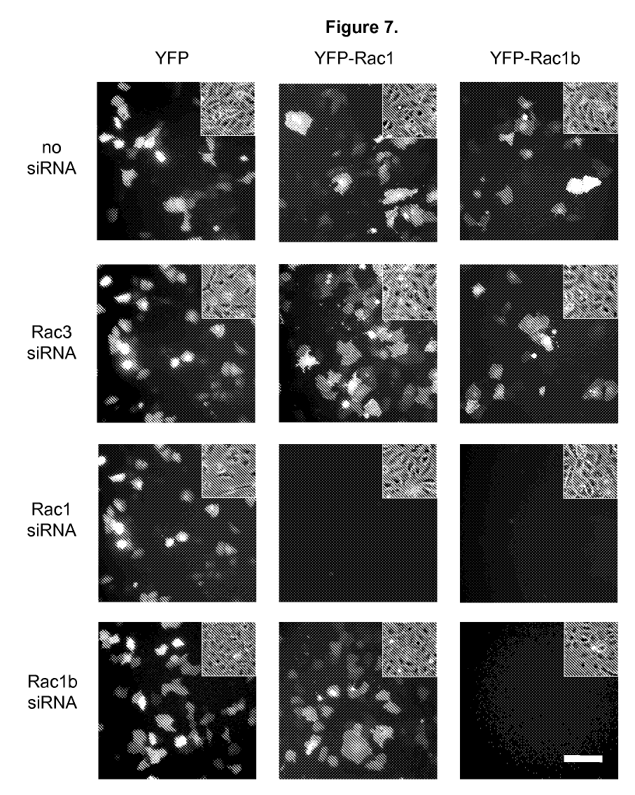

Previous experiments had shown that exposure of mammary epithelial cells to MMP-3 caused increased cell motility, invasiveness, and progression to malignancy, all characteristics of the epithelial-mesenchymal transition (Stemlicht, M. D. et al. The stromal proteinase MMP3/stromelysin-1 promotes mammary carcinogenesis. Cell 98, 137-46, 1999; Lochter, A. et al. Misregulation of stromelysin-1 expression in mouse mammary tumor cells accompanies acquisition of stromelysin-1-dependent invasive properties. J Biol Chem 272, 5007-15, 1997; Lochter, A. et al. Matrix metalloproteinase stromelysin-1 triggers a cascade of molecular alterations that leads to stable epithelial-to-mesenchymal conversion and a premalignant phenotype in mammary epithelial cells. J Cell Biol 139, 1861-72, 1997. Here we show that this occurs through the induction of Rac1b, a highly activated splice isoform of Rac1. MMP-3-mediated induction of Rac1b (FIG. 1b-e, FIG. 9) causes alterations in the actin cytoskeleton (FIG. 1a), increased motility (FIG. 1f), and altered gene expression characgteristic or epithelial-mesenchymal transition (FIG. 5). Inhibition of Rac1b by siRNA selectively blocked the effects of MMP-3 (FIG. 1g-h, FIG. 7).

Referring now to FIG. 1a, MMP-3 induces epithelial-mesenchymal transition (EMT) through Rac1b as shown by MMP-3-induced alterations in actin cytoskeleton. These photographs (scale bar, 25 μm) show that untreated mammary epithelial cells retain their normal characteristics, while MMP-3 treated mammary epithelial cells develop lamellipodia and increased cell motility which are signs of increased invasiveness and progression to malignancy.

Analysis of active and total levels of Rac shown in the gel in FIG. 1b show that the induction of Rac1b occurs after MMP-3 treatment. The methods described in Example 1 were used to show Rac1 and Rac1b transcript levels by RT/PCR (FIG. 1c) and measure Rac1b protein expression through the use of an antibody raised against the mouse Rac1b insertion sequence (FIG. 1d). As shown in FIGS. 1b-1d, the treatment of mammary epithelial cells with MMP-3 upregulates Rac1b transcript and expression levels. Referring now to the graph in FIG. 1e, Rac1b transcript levels are shown to increase in response to MMP-3 treatment (days 1-4) and decrease during washout (days 5-6). The blue diamonds indicate transcript levels of MMP-3 treated cells as measured by Rac1b/GADPH and the red squares indicate the transcript levels of untreated cells.

FIG. 1f is a graph showing rate of cell migration and motility as assessed by scratch assay (Goodman, S. L., Vollmers, H. P., and Birchmeier, W. Cell 41, 1029-1038, 1985). As shown in the graph, treatment with MMP-3 and Rac1b causes an increase in cell migration and motility, both hallmarks of malignant transformation. MMP-3 treatment of cells blocked with Rac1N17 exhibit only a slight increase in motility as compared to normal untreated cells.

Referring to FIG. 5a, MMP-3-treated SCp2 cells, stained for cytokeratins (red), vimentin (green), and DNA (blue) (scale bar, 50 μm) show an increase in vimentin and decrease in cytokeratins over the course of treatment with MMP-3 for 28 days. On the 7^{th} day, some cells a minority of cells stained for vimentin, with the majority stained red or deep orange (mixture of both antibody stains). At the 14^{th} day, half the cells stain completely for vimentin, while the other half stain for both cytokeratins and vimentin. However, by the 28^{th} day, the cells stain completely green for vimentin.

Other cancer marker transcript levels were measured in cells treated with MMP-3 for 4 days (p<0.001 for all altered expression levels) (FIG. 5b). Transcript levels of keratin, E-cadherin, vimentin, SM actin, TGFβ, Snail, collagen A1 and fibronectin were measured for altered expression levels. All markers except for keratin and E-cadherin showed a two-fold or greater increase in expression levels. Keratin and E-cadherin showed a greater than two-fold decrease in expression levels.

Lastly, vimentin transcript levels were measured in response to MMP-3 treatment (days 1-4) and washout (days 5-6) (blue diamonds, treated; red squares, untreated; p<0.001 for day 4 treated vs. either day 1 treated or day 4 untreated). MMP-3 treatment causes an increase in vimentin transcript levels, which can be reversed upon washout.

We tested to see if the siRNAs obtained could carry out selective knockdown of cotransfected constructs. Referring now to FIG. 7, we found that Rac1 siRNA blocked expression of cotransfected and YFP-Rac1b, and that the specific Rac1b siRNA blocked expression of only cotransfected YFP-Rac1b, not YFP-Rac1; none of the siRNAs affected expression of cotransfected YFP. The effect on endogenous gene expression levels was consistent with effective knockdown of all transiently transfected cells (˜70% transfection efficiency), and showed that Rac1 siRNA inhibits expression of both Rac1 and Rac1b but does not affect Rac3, Rac1b siRNA (SEQ ID NO:4) selectively targets Rac1b and does not affect Rac1 or Rac3, and Rac3 siRNA selectively targets Rac3 and does not affect expression of Rac1 or Rac1b. FIG. 1g shows the quantification of knockdown of endogenous gene expression. When SCp2 cells were transiently cotransfected with YFP and either no siRNA, or siRNA targeting Rac3, Rac1/Rac1b, or Rac1b, and then treated with MMP-3 for 4 days, we observed that siRNA for Rac1/Rac1b or Rac1b inhibited MMP-3-induced cell motility in the cotransfected colonies, while siRNA targeting Rac3 had no effect (FIG. 1h).

### Example 3

### MMP-3/Rac1b Stimulates Mitochondrial Production of ROS

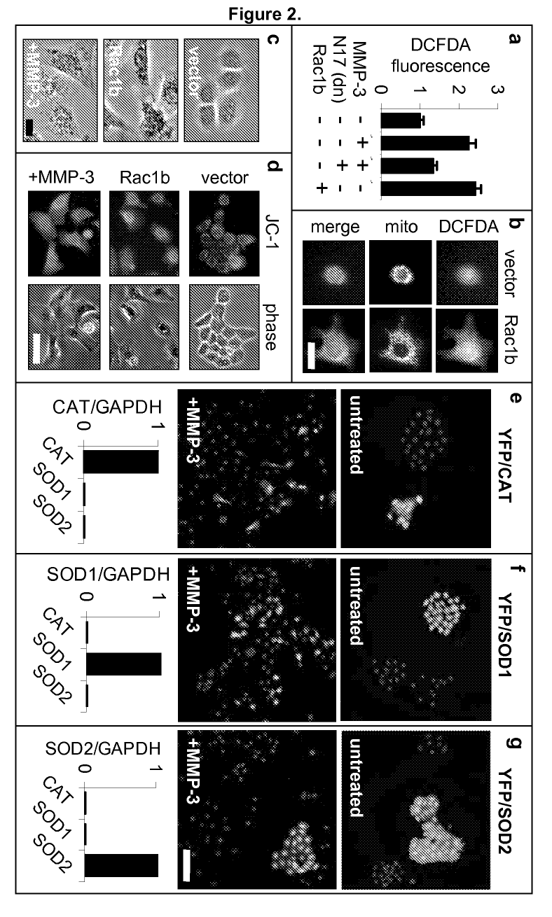

The Rac1b-induced changes in the cell skeleton also stimulated the formation of extremely active molecules known as reactive oxygen species, or ROS. MMP-3/Rac1b stimulate mitochondrial production of ROS. Increased cellular ROS levels in MMP-3-treated or Rac1b-expressing cells was measured by increased DCFDA fluorescence (FIG. 2a). Identification of the mitochondria as the source of the MMP-3/Rac1b-induced ROS was determined by localization of DCFDA fluorescence (FIG. 2b), ROS-mediated precipitation of nitrobluetetrazolium in a mitochondrial pattern (FIG. 2c), and induced depolarization of mitochondria, as shown by loss of red JC-1 fluorescence (FIG. 2d).

Furthermore, specific inhibition of mitochondrial ROS by expression of mitochondrial superoxide dismutase (SOD2) blocked the MMP-3-induced effects (FIG. 2g), while the expression of cytosolic ROS-quenching enzymes catalase (CAT; FIG. 2e) and superoxide dismutase 1 (SOD 1; FIG. 2f) had no effect (FIG. 2e-f). Cells were cotransfected with EYFP and catalase, SOD1 or SOD2) and then cultured in the absence (upper image) or presence (lower image) of MMP-3 for 6 days. EYFP fluorescence, green; nuclei, red; graphs at bottom show gene transcript levels in transfected cell populations; scale bar, 100 μm.

### Example 4

### MMP-3-Induced EMT Is Dependent Upon ROS And Rac1 Activity

MMP-3/Rac1b-induced reactive oxygen species (ROS) activate a variety of transcription factors. One transcription factor identified as activated by the Rac1b-induced ROS was Snail, which has been studied as a master regulator of the epithelial-mesenchymal transition²⁴.

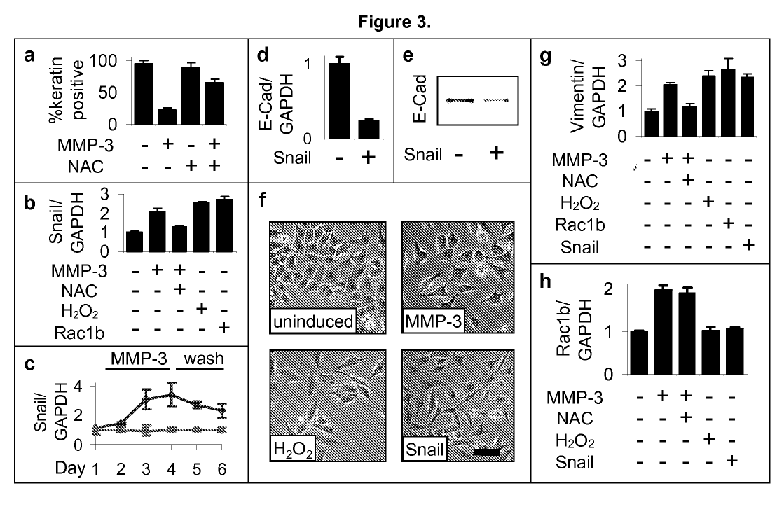

MMP-3, Rac1b, or ROS were sufficient to activate Snail, and inhibition of ROS blocked MMP-3 or Rac1b-mediated activation of Snail, but not Snail-mediated EMT (FIG. 3 a,b,c,g,h). These experiments established Snail as downstream of MMP-3/Rac1b-mediated activation of ROS.

We determined that MMP-3 enhances expression of the transcription factor Snail (Nieto, M. A. The snail superfamily of zinc-finger transcription factors. Nat Rev Mol Cell Biol 3, 155-66, 2002; Thiery, J. P. Epithelial-mesenchymal transitions in tumour progression. Nat Rev Cancer 2, 442-54, 2002), and that this effect could be blocked by treatment with NAC, or induced in the absence of MMP-3 by elevating ROS levels with H₂O₂ or by expression of Rac1b. Referring now to FIG. 3a, NAC inhibits MMP-3-induced downregulation of epithelial cytokeratin protein levels. Induction of Snail occurs by MMP-3, and in the absence of MMP-3 by elevating ROS levels with H₂O₂ or by expression of Rac1b (FIG. 3b). FIG. 3c shows Snail transcript levels in response to MMP-3 treatment (days 1-4) and washout (days 5-6), with the blue diamonds indicated treated cells and the red squares indicating untreated.

Snail was shown to mediate epithelial-mesenchymal transition in the experimental system comparable to that induced by ROS or EMT (FIG. 3d-f). Expression of Snail in SCp2 cells was sufficient to induce EMT: induction caused downmodulation of E-cadherin transcript and protein levels (FIG. 3d,e) and led to cell scattering comparable to that induced by MMP-3 or H₂O₂ (FIG. 3f). In FIGS. 3d-e, exogenous expression of Snail in SCp2 cells reduces E-cadherin transcript (d) and protein levels (e). FIG. 3f shows photographs of cell scattering induced by treatment with MMP-3 or H₂O₂ or by exogenous expression of Snail; scale bar 50 μm.

We also found that while MMP-3, Rac1b, H₂O₂, or Snail can stimulate expression of mesenchymal vimentin (FIG. 3g), only MMP-3 could stimulate expression of Rac1b (FIG. 3h). When combined with the data presented in Examples 2 and 3 and FIGS. 1b-f and 2a-d, these results show that treatment with MMP-3 stimulates expression of Rac1b, which causes increases in cellular ROS, leading in turn to increased expression of Snail and EMT.

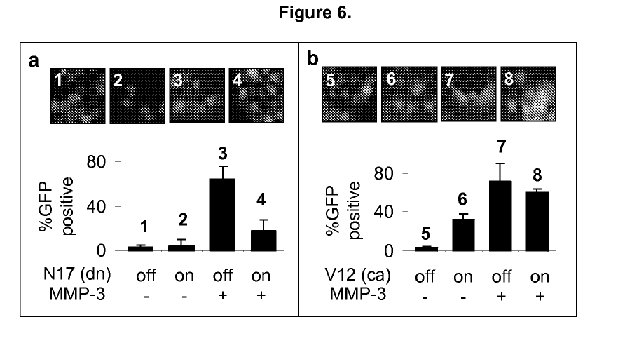

An essential role for Rac1 activity in MMP-3-mediated EMT was also shown by the fact that expression of dominant negative Rac1 blocked the MMP-3-mediated effects, while expression of constitutively active mutant Rac1 reproduced these effects (FIG. 6). Rac1-dependence was tested using tetracycline-regulated adenoviral expression vectors and a vimentin promoter reporter system (courtesy C. Gilles, University of Liege, Belgium). Activation of vimentin promoter by treatment with MMP-3 (4 d) is attenuated by inducible expression of dominant negative (dn) Rac1N17 (FIG. 6a), whereas inducible expression of consititutively active (ca) Rac1V12 (4 d) is sufficient to activate vimentin promoter even in the absence of MMP-3 (FIG. 6b); insets show sample images of indicated experiments (green, GFP; red, nuclei).

### Example 5

### MMP-3-Induced ROS Causes DNA Damage And Genomic Instability

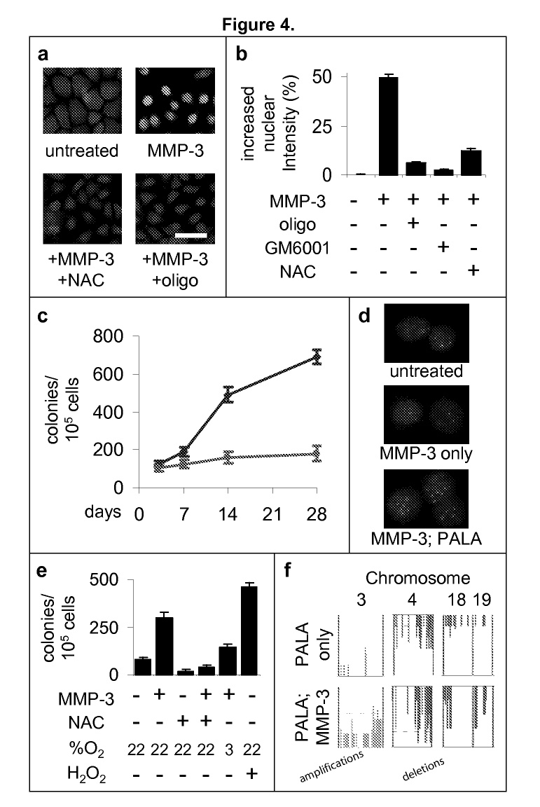

Damage of DNA often results in loss of genomic integrity, resulting in increases and decreases in chromosome content. To test for DNA damage, we used fluorescein isothiocyanate (FITC)-conjugated avidin, as this reagent binds to 8-oxodeoxyguanosine, an oxidative DNA lesion with structural similarity to biotin (Struthers, L., Patel, R., Clark, J. & Thomas, S. Direct detection of 8-oxodeoxyguanosine and 8-oxoguanine by avidin and its analogues. Anal Biochem 255, 20-31, 1998). Cells treated with MMP-3 showed significantly increased FITC-avidin nuclear staining (FIG. 4a) that was blocked by preincubating with an oligonucleotide containing 8-oxodeoxyguanosine (but not with a control oligonucleotide; not shown), by inhibiting the proteolytic activity of MMP-3 with GM6001, or by treatment with NAC. Thus, MMP-3-induced ROS were shown to directly damage DNA by increased nuclear fluorescence of cells incubated with 8-oxoguanosine (FIG. 4a-b). FIG. 4b shows a quantification of increased nuclear staining in MMP-3 treated cells relative to untreated (error bars, 95% CI).

To test for induction of genomic instability, we assayed for increased resistance of MMP-3-treated SCp2 mouse mammary epithelial cells to N-(phosphonacetyl)-L-aspartate (PALA) (Johnson, R. K., Inouye, T., Goldin, A. & Stark, G. R. Antitumor activity of N-(phosphonacetyl)-L-aspartic acid, a transition-state inhibitor of aspartate transcarbamylase. Cancer Res 36, 2720-25, 1976), since resistance to PALA is acquired through amplification of the CAD gene (Wahl, G. M., Padgett, R. A. & Stark, G. R. Gene amplification causes overproduction of the first three enzymes of UMP synthesis in N-(phosphonacetyl)-L-aspartate-resistant hamster cells. J Biol Chem 254, 8679-89, 1979). Exposure to MMP-3 led to a time-dependent increase in the fraction of cells that had acquired PALA resistance (FIG. 4c) that was due to amplification of the CAD locus (FIG. 4d). A graph showing the increase in fractions of cells acquiring PALA resistance by MMP-3 is shown in FIG. 2c (blue diamonds, MMP-3; red squares, untreated). The number of colonies per 10⁵ cells increases noticeably after one week of MMP-3 exposure from 200 colonies/10⁵ cells to 700 colonies/10⁵ cells after 28 days. Fluorescence in situ hybridization of the CAD gene locus (red spots in cells) confirms the increase in genomic amplification of CAD (FIG. 4d).

MMP-3-induced ROS were shown to cause genomic amplification of the CAD locus by increased resistance to PALA and assessment of CAD locus in MMP-3-treated cells (FIG. 4c-e). This effect also could be inhibited by treatment with NAC or by culturing under reduced oxygen tension, and reproduced in the absence of MMP-3 by treatment with H₂O₂ (FIG. 4e). That the genomic instability induced by MMP-3 was not limited to the CAD locus was shown by CGH analysis, as many additional genomic amplifications and deletions were found in MMP-3-treated cells (FIG. 4f), including characteristic alterations previously observed in tumors derived from the MMP-3 transgenic mice (Stemlicht, M. D. et al. The stromal proteinase MMP3/stromelysin-1 promotes mammary carcinogenesis. Cell 98, 137-46, 1999). Frequency plots of comparative genomic hybridization CGH analyses of cells grown in the absence (top) or presence (bottom) of MMP-3, and then selected with PALA show widespread chromosome amplifications and deletions were found in the MMP-3-treated cells (FIG. 4f).

### Example 6

### The Effects of MMP-3 Are Specifically To Its Proteolytic Activity

To verify that the effects of MMP-3 were due to its proteolytic activity, a mutant inactive form of MMP-3 was generated (MMP-3EA) and shown to lack the effects of the normal protein (FIG. 8a). Comparing the images of uninduced cells with those of catalytically inactive MMP-3 (MMP-3EA), it can be seen that MMP-3EA does not induce EMT, but also does not block EMT induced by active MMP-3. Scale bar, 50 μm. The inset graph shows the MMP-3EA expression in uninduced and induced cells, analyzed by quantitative RT/PCR and normalized to GAPDH expression. Error bars, SEM; p<0.001 for comparison.

The MMP inhibitor GM6001 blocked the MMP-3-mediated effects. FIG. 8b shows that activation of vimentin-EGFP construct and effect of MMP inhibitor (GM6001) on cells treated with MMP-3. Scale bar, 50 μm. MMP-3 induced cells exhibit the flattened morphology of invasive cells, but coincubation with GM6001 prevents the malignant transformation.

It is clear that the extracellular proteolytic activity of MMP-3 is essential (FIG. 8). We have shown that MMP-3 effectively cleaves E-cadherin, resulting in loss of cell-cell adhesions and relocalization of transcriptionally active β-catenin to the nucleus (See, Lochter, A. et al. J Biol Chem 272, 5007-15, 1997; Lochter, A. et al. J Cell Biol 139, 1861-72, 1997; and not shown). It is important to note that MMP-3 is not the only protease capable of initiating this pathway, as we have found that MMP-9 (but not MMP-2) can substitute for MMP-3 in our experimental system (not shown), and MMP-7 and MMP-14 are also known to induce tumors when expressed in transgenic mice (Sternlicht, M. D. & Werb, Z. How matrix metalloproteinases regulate cell behavior. Annu Rev Cell Dev Biol 17, 463-516, 2001). Furthermore, MMPs are not the only microenvironmental components implicated in tumor induction or progression, as oncogenic properties have also been attributed to TGFβ, growth factors, and hormones, and the tumor-promoting activities of chronic inflammation are well known (Bissell, M. J. & Radisky, D. Putting tumours in context. Nat Rev Cancer 1, 46-54, 2001). Our investigations of MMP-3 show how this factor can directly stimulate phenotypic and genotypic malignant transformation in normally functioning cells. We expect that similar or parallel pathways may be induced by other elements of the tumor microenvironment, and we suspect that such mechanisms may be much more relevant for generation of genomic instability than predicted by current models of tumor progression.

### Example 7

### Effect of YFP-Fused Rac1 And Rac1b Constructs On Cell Morphology

Validation of mouse Rac1b as being a highly active form was tested by expressing YFP-fused constructs of Rac1b, as well as the constitutively active Rac1V12 and the dominant negative Rac1N17. Mouse Rac1b was cloned from cDNA derived from MMP-3-treated cells expressed as a fusion with YFP; endogenous mouse Rac1 was also cloned and used to generate active YFPRac1V12 and inhibitory YFPRac1N17 constructs. These constructs were assessed by assessing altered cytoskeletal morphology. FIG. 10 shows the effect of YFP-fused Rac1 and Rac1b constructs on cell morphology. The images on the left show the MMP-3 treated cells expressing the indicated constructs with actin stained with Texas red phalloidin. The images on the right, show YFP staining. MMP-3-treated cells expressing only YFP have normal cell morphology as do cells expressing inhibitory YFPRac1N17 constructs. MMP-3-treated cells expressing YFP and mouse Rac1 and active YFPRac1V12 exhibited abnormal morphology similar to that of invasive cells.

The constructs were further assessed for their catalytic activity by association with PAK (FIG. 11). The western blot of the activity assay of YFP-fused mouse Rac1b and Rac1V12 shows that both exhibit similar activity.

Induction of epithelial-mesenchymal transition (EMT) by treatment of SCp2 mouse mammary epithelial cells with MMP-3 is associated with loss of intact E-cadherin, increased motility and invasiveness, downmodulation of epithelial markers, and upregulation of mesenchymal markers (See, Lochter, A. et al. J Biol Chem 272, 5007-15, 1997; Lochter, A. et al. J Cell Biol 139, 1861-72, 1997; and FIG. 5a,b), through a process that is initially reversible (See, Lochter, A. et al. J Biol Chem 272, 5007-15, 1997; Lochter, A. et al. J Cell Biol 139, 1861-72, 1997; and FIG. 5c). The MMP-3-induced morphological alteration of the F-actin cytoskeleton suggested the involvement of members of the Rho GTPase family (FIG. 1a), and while the activity of RhoA and Cdc42 were unchanged (not shown), we were intrigued by an additional band in the Rac activity assay of MMP-3-treated cells (FIG. 1b). A highly activated splice isoform of Rac1, designated Rac1b, containing 57 additional nucleotides that result in an in-frame insertion of 19 additional amino acids was discovered recently in breast and colorectal tumors (Schnelzer, A. et al. Rac1 in human breast cancer: overexpression, mutation analysis, and characterization of a new isoform, Rac1b. Oncogene 19, 3013-20, 2000; Jordan, P., Brazao, R., Boavida, M. G., Gespach, C. & Chastre, E. Cloning of a novel human Rac1b splice variant with increased expression in colorectal tumors. Oncogene 18, 6835-39, 1999) and has transforming characteristics when exogenously expressed in cultured cells (Singh, A. et al. Rac1b, a tumor associated, constitutively active Rac1 splice variant, promotes cellular transformation. Oncogene 23, 9369-80, 2004). We identified the additional Rac band induced by MMP-3 as Rac1b by RT/PCR (FIG. 1c) and through the use of an antibody raised against the mouse Rac1b insertion sequence (FIG. 1d); we also found that induction of Rac1b by treatment with MMP-3 was initially reversible (FIG. 1e). We determined that the activity of Rac1b was required for the MMP-3-induced alterations in vimentin expression (FIG. 6), and for MMP-3-induced motility (FIG. 1f), as dominant negative Rac1N17 attenuated the effects of MMP-3, and expression of Rac1b could substitute for MMP-3 (FIG. 1f).

We also evaluated the relationship between induction of Rac1b and downstream EMT by specific transcript knockdown using small interfering RNA (siRNA). SCp2 cells were cotransfected transiently with yellow fluorescent protein (YFP), YFP-Rac1, or YFP-Rac1b, and either no siRNA, siRNA targeting Rac3, siRNA targeting Rac1 (which also targets Rac1b) or siRNA selectively targeting the splice insertion sequence in Rac1b (these cells do not express Rac2). The sequences of the siRNA targeting Rac3 and Rac1 are unknown. The siRNAs selectively targeting the splice insertion sequence in Rac1b were comprised of the following sequences:

| | |
|---|---|
| SEQ ID NO: 1 | CACAUGUGGUAAAGAUAGA |
| SEQ ID NO: 2 | ACAAGCCGAUUGCCGACGUGUUC |
| SEQ ID NO: 3 | GACAGUUGGAGACACAUGUGGUAAA |
| SEQ ID NO: 4 | UGGAGACACAUGUGGUAAAGAUAGA |

We found that Rac1 siRNA blocked expression of cotransfected and YFP-Rac1b, and that the specific Rac1b siRNA blocked expression of only cotransfected YFP-Rac1b, not YFP-Rac1 (FIG. 7); none of the siRNAs affected expression of cotransfected YFP. The effect on endogenous gene expression levels was consistent with effective knockdown of all transiently transfected cells (˜70% transfection efficiency), and showed that Rac1 siRNA inhibits expression of both Rac1 and Rac1b but does not affect Rac3, Rac1b siRNA (SEQ ID NO: 4) selectively targets Rac1b and does not affect Rac1 or Rac3, and Rac3 siRNA selectively targets Rac3 and does not affect expression of Rac1 or Rac1b (FIG. 1g). When SCp2 cells were transiently cotransfected with YFP and either no siRNA, or siRNA targeting Rac3, Rac1/Rac1b, or Rac1b, and then treated with MMP-3 for 4 days, we observed that siRNA for Rac1/Rac1b or Rac1b inhibited MMP-3-induced cell motility in the cotransfected colonies, while siRNA targeting Rac3 had no effect (FIG. 1h).

We show that increased Rac activity leads to the diverse alterations induced by MMP-3. Previous studies (Kheradmand, F., Werner, E., Tremble, P., Symons, M. & Werb, Z. Role of Rac1 and oxygen radicals in collagenase-1 expression induced by cell shape change. Science 280, 898-902, 1998; Werner, E. & Werb, Z. Integrins engage mitochondrial function for signal transduction by a mechanism dependent on Rho GTPases. J Cell Biol 158, 357-68, 2002) showed that active Rac can stimulate production and release of mitochondrial superoxide into the cytoplasm. Excess superoxide production can cause oxidative DNA damage and genomic instability (Samper, E., Nicholls, D. G. & Melov, S. Mitochondrial oxidative stress causes chromosomal instability of mouse embryonic fibroblasts. Aging Cell 2, 277-85, 2003), transform cells in culture (Suh, Y. A. et al. Cell transformation by the superoxide-generating oxidase Mox1. Nature 401, 79-82, 1999), and potentiate tumor progression (Droge, W. Free radicals in the physiological control of cell function. Physiol Rev 82, 47-95, 2002), and superoxide is readily converted to other forms of ROS that stimulate additional tumorigenic processes (Droge, W. Free radicals in the physiological control of cell function. Physiol Rev 82, 47-95, 2002; Puri, P. L. et al. A myogenic differentiation checkpoint activated by genotoxic stress. Nat Genet 32, 585-93, 2002; Finkel, T. Oxidant signals and oxidative stress. Curr Opin Cell Biol 15, 247-54, 2003). We found that treatment with MMP-3 or expression of Rac1b produced increases in cellular ROS, as assessed by the fluorophore dichlorodihydrofluorescein diacetate (DCFDA), and that expression of Rac1N17 attenuated the induction of ROS by MMP-3 (FIG. 2a). The DCFDA fluorescence partially colocalized with a mitochondrial marker protein (FIG. 2b), and the identity of the induced ROS as mitochondrial superoxide was indicated by the staining pattern of nitrobluetetrazolium (FIG. 2c), which forms an insoluble blue formazan in the presence of superoxide (Werner, E. & Werb, Z. Integrins engage mitochondrial function for signal transduction by a mechanism dependent on Rho GTPases. J Cell Biol 158, 357-68, 2002), and by the altered fluorescence pattern of cells stained with JC-1, in which the punctate red mitochondrial staining of the J-aggregate of JC-1 is replaced by diffuse cytoplasmic green staining of the monomeric form (FIG. 2d), consistent with dissipation of membrane potential following mitochondrial production of superoxide (Werner, E. & Werb, Z. J Cell Biol 158, 357-68, 2002; Madesh, M. & Hajnoczky, G. VDAC-dependent permeabilization of the outer mitochondrial membrane by superoxide induces rapid and massive cytochrome c release. J Cell Biol 155, 1003-15, 2001). To determine whether the induction of mitochondrial superoxide by MMP-3/Rac1b was essential for the induction of EMT, we cotransfected cells with expression plasmids encoding YFP and either catalase (CAT), superoxide dismutase-1 (SOD 1), or SOD2. CAT stimulates the decomposition of H₂O₂ into water and molecular oxygen, while SOD1 and SOD2 convert superoxide into H₂O₂ and molecular oxygen; CAT and SOD1 are cytoplasmic enzymes, while SOD2 is localized to the mitochondria. These experiments demonstrated that YFP/SOD2 cells were resistant to MMP-3-induced scattering (FIG. 2g), while YFP/CAT and YFP/SOD1 cells responded in a similar fashion to adjacent untransfected cells (FIG. 2e,f).

ROS can alter gene expression (Droge, W. Free radicals in the physiological control of cell function. Physiol Rev 82, 47-95, 2002; Puri, P. L. et al. A myogenic differentiation checkpoint activated by genotoxic stress. Nat Genet 32, 585-93, 2002; Finkel, T. Oxidant signals and oxidative stress. Curr Opin Cell Biol 15, 247-54, 2003) and stimulate cell invasiveness (Mori, K., Shibanuma, M. & Nose, K. Invasive potential induced under long-term oxidative stress in mammary epithelial cells. Cancer Res 64, 7464-72, 2004), and we found that the ROS-quenching agent N-acetyl cysteine (NAC) effectively inhibited MMP-3-induced downregulation of epithelial cytokeratins (FIG. 3a) and upregulation of mesenchymal vimentin (FIG. 3g). NAC also inhibited MMP-3-induced cell motility, invasion, and morphological alterations (not shown). Induction of EMT involves the coordinated regulation of many genes (Kalluri, R. & Neilson, E. G. Epithelial-mesenchymal transition and its implications for fibrosis. J Clin Invest 112, 1776-84, 2003); here, we focused on MMP-3/ROS-dependent alterations in the expression levels of transcriptional regulatory proteins that mediate EMT. We determined that MMP-3 enhances expression of the transcription factor Snail (Nieto, M. A. The snail superfamily of zinc-finger transcription factors. Nat Rev Mol Cell Biol 3, 155-66, 2002; Thiery, J. P. Epithelial-mesenchymal transitions in tumour progression. Nat Rev Cancer 2, 442-54, 2002), and that this effect could be blocked by treatment with NAC, or induced in the absence of MMP-3 by elevating ROS levels with H₂O₂ or by expression of Rac1b (FIG. 3b). Expression of Snail in SCp2 cells was sufficient to induce EMT: induction caused downmodulation of E-cadherin transcript and protein levels (FIG. 3d,e) and led to cell scattering comparable to that induced by MMP-3 or H₂O₂ (FIG. 4f). We also found that while MMP-3, Rac1b, H₂O₂, or Snail can stimulate expression of mesenchymal vimentin (FIG. 3g), only MMP-3 could stimulate expression of Rac1b (FIG. 3h). When combined with the data presented in FIGS. 1b-f and 2a-d, these results show that treatment with MMP-3 stimulates expression of Rac1b, which causes increases in cellular ROS, leading in turn to increased expression of Snail and EMT.

We previously had found that tumors in the MMP-3-expressing transgenic mice showed common patterns of genomic rearrangements (Sternlicht, M. D. et al. The stromal proteinase MMP3/stromelysin-1 promotes mammary carcinogenesis. Cell 98, 137-46, 1999), suggesting that MMP-3 could lead to genomic instability in target epithelial cells in vivo. Given the known genotoxic effects of ROS, we investigated the effects of MMP-3-induced ROS on the integrity of the genome under defined conditions in culture. To test for DNA damage, we used fluorescein isothiocyanate (FITC)-conjugated avidin, as this reagent binds to 8-oxodeoxyguanosine, an oxidative DNA lesion with structural similarity to biotin (Struthers, L., Patel, R., Clark, J. & Thomas, S. Direct detection of 8-oxodeoxyguanosine and 8-oxoguanine by avidin and its analogues. Anal Biochem 255, 20-31, 1998). Cells treated with MMP-3 showed significantly increased FITC-avidin nuclear staining (FIG. 4a) that was blocked by preincubating with an oligonucleotide containing 8-oxodeoxyguanosine (but not with a control oligonucleotide; not shown), by inhibiting the proteolytic activity of MMP-3 with GM6001, or by treatment with NAC (FIG. 4b). To test for induction of genomic instability, we assayed for increased resistance of MMP-3-treated SCp2 mouse mammary epithelial cells to N-(phosphonacetyl)-L-aspartate (PALA) (Johnson, R. K., Inouye, T., Goldin, A. & Stark, G. R. Antitumor activity of N-(phosphonacetyl)-L-aspartic acid, a transition-state inhibitor of aspartate transcarbamylase. Cancer Res 36, 2720-5, 1976), since resistance to PALA is acquired through amplification of the CAD gene (Wahl, G. M., Padgett, R. A. & Stark, G. R. Gene amplification causes overproduction of the first three enzymes of UMP synthesis in N-(phosphonacetyl)-L-aspartate-resistant hamster cells. J Biol Chem 254, 8679-89, 1979). Exposure to MMP-3 led to a time-dependent increase in the fraction of cells that had acquired PALA resistance (FIG. 4c) that was due to amplification of the CAD locus (FIG. 4d). This effect also could be inhibited by treatment with NAC or by culturing under reduced oxygen tension, and reproduced in the absence of MMP-3 by treatment with H₂O₂ (FIG. 4e). That the genomic instability induced by MMP-3 was not limited to the CAD locus was shown by CGH analysis, as many additional genomic amplifications and deletions were found in MMP-3-treated cells (FIG. 4f), including characteristic alterations previously observed in tumors derived from the MMP-3 transgenic mice (Sternlicht, M. D. et al. Cell 98, 137-46, 1999).

Our results show that a key event in MMP-3-induced malignant transformation of SCp2 cells is the induction of Rac1b, an alternative splice isoform of Rac1 that was initially identified in breast and colon cancers (Schnelzer, A. et al. Rac1 in human breast cancer: overexpression, mutation analysis, and characterization of a new isoform, Rac1b. Oncogene 19, 3013-20, 2000; Jordan, P., Brazao, R., Boavida, M. G., Gespach, C. & Chastre, E. Cloning of a novel human Rac1b splice variant with increased expression in colorectal tumors. Oncogene 18, 6835-9, 1999). Many oncogenic splice isoforms are induced in cancers (Shin, C. & Manley, J. L. Cell signalling and the control of pre-mRNA splicing. Nat Rev Mol Cell Biol 5, 727-38, 2004), and although most of these produce proteins that lack key functional domains, Rac1b is unusual in that it becomes more highly activated (Matos, P., Collard, J. G. & Jordan, P. Tumor-related alternatively spliced Rac1b is not regulated by Rho-GDP dissociation inhibitors and exhibits selective downstream signaling. J Biol Chem 278, 50442-8, 2003; Fiegen, D. et al. Alternative splicing of Rac1 generates Rac1b, a self-activating GTPase. J Biol Chem 279, 4743-9, 2004). The fact that Rac1b is the only apparent splice isoform of Rac1 found in MMP-3-treated cells is significant, since Rac1b is also the only apparent splice isoform in breast cancer cells (Schnelzer, A. et al. Oncogene 19, 3013-20 (2000). MMP-3 treatment leads to alternative splicing of Rac1b.

Our results show that Rac1b expression and its elevated expression induces matrix metalloproteinase activity, which can cause epithelial-mesenchymal transition (EMT) and malignant transformation. Rac1b expression also is shown herein to induce the activation of reactive oxygen species (ROS), which can cause genomic instability, thereby also promoting malignant transformation of cells. Therefore the detection and inhibition of Rac1b expression may be useful in early detection and treatment of cancer.

While the present sequences, compositions and processes have been described with reference to specific details of certain exemplary embodiments thereof, it is not intended that such details be regarded as limitations upon the scope of the invention. The present examples, methods, procedures, specific compounds and molecules are meant to exemplify and illustrate the invention and should in no way be seen as limiting the scope of the invention. Any patents, patent applications, publications, Genbank Accession Nos., publicly available sequences mentioned in this specification and below are indicative of levels of those skilled in the art to which the invention pertains and are hereby incorporated by reference to the same extent as if each was specifically and individually incorporated by reference.

We claim:

1. A method for inhibiting matrix metalloproteinase (MMP) induced malignant transformation of a cell, said method comprising contacting a cell with a compound that modulates Rac1b.

2. The method of claim 1, wherein the MMP is selected from the group consisting of MMP-3 and MMP-9.

3. The method of claim 1, wherein the compound comprises an siRNA molecule that selectively inhibits expression of Rac1b.

4. The method of claim 3, wherein the siRNA molecule comprises a sequence selected from the group consisting of: SEQ ID NOS: 1, 2, 3, and 4.

5. The method of claim 1, wherein the compound comprises an antibody that specifically binds to Rac1b.

6. The method of claim 5, wherein the antibody is a monoclonal antibody.

7. The method of claim 5, wherein the antibody is humanized.

8. The method of claim 5, wherein the antibody is a member selected from the group consisting of: a Fab fragment, a Fv fragment, a scFv, and combinations thereof.

9. The method of claim 5, wherein the antibody specifically binds to a polypeptide encoded by a sequence selected from the group consisting of: SEQ ID NOS:5, 8 and subsequences thereof.

10. The method of claim 5, wherein the antibody specifically binds to a polypeptide comprising a sequence selected from the group consisting of: SEQ ID NOS: 6, 9, and subsequences thereof.

11. The method of claim 1, wherein the cell is in a mammal.

12. The method of claim 11, wherein the mammal is a human.

13. The method of claim 12, wherein the human has been diagnosed with MMP-associated cancer, wherein the cancer is selected from the group consisting of: breast cancer, lung cancer, prostate cancer, pancreatic cancer, ovarian cancer, metastatic melanoma, uroepithelial cancer, invasive oral cancer, gastric cancer, and head and neck squamous cell carcinoma.

14. The method of claim 13, wherein the MMP is selected from the group consisting of MMP-3 and MMP-9.

15. A method for detecting MMP induced malignancy by detecting expression of Rac1b, said method comprising detecting the sequence set forth in SEQ ID NOS:5, 6, 8, 9 or a subsequence thereof.

16. The method of claim 15, wherein said detecting comprises;
(a) contacting a sample with an oligonucleotide that selectively hybridizes to a nucleic acid sequence selected from the group consisting of: SEQ ID NOS:5, 8 and subsequences thereof under conditions sufficient for the oligonucleotide to form a complex with the sequence;
(b) determining whether a complex forms between the oligonucleotide and the sequence; and
(c) detecting expression of Rac1b by detecting the complex of step (b), whereby expression of Rac1b detects the MMP induced malignancy.

17. The method of claim 15, wherein said detecting comprises:
(a) contacting a sample with primers that specifically amplify a nucleic acid sequence comprising a sequence selected from the group consisting of: SEQ ID NOS:5, 8 and subsequences thereof, under conditions sufficient to amplify the sequence;
(b) determining whether an amplification product is formed; and
(c) detecting expression of Rac1b by detecting the amplification product of step (b), whereby expression of Rac1b detects the MMP-3 induced malignancy.

18. The method of claim 16 or 17, wherein the sample is from a mammal suspected of having MMP induced cancer.

19. The method of claim 18, wherein the mammal is a human.

20. The method of claim 15, wherein said detecting comprises
(a) contacting a sample with an antibody that specifically binds to a polypeptide comprising a sequence selected from the group consisting of: SEQ ID NO: 6, 9, and subsequences thereof under conditions sufficient for the antibody form a complex with the polypeptide,
(b) determining whether a complex forms between the antibody and the polypeptide; and
(c) detecting expression of Rac1b by detecting the complex of step (b), whereby expression of Rac1b detects the MMP induced malignancy.

21. The method of claim 15, wherein said detecting comprises
(a) contacting a sample with an antibody that specifically binds to a polypeptide comprising a sequence encoded by a sequence selected from the group consisting of: SEQ ID NO: 5, 8, and subsequences thereof, under conditions sufficient for the antibody form a complex with the polypeptide,
(b) determining whether a complex forms between the antibody and the polypeptide; and
(c) detecting expression of Rac1b by detecting the complex of step (b), whereby expression of Rac1b detects the MMP induced malignancy.

22. The method of claim 20 or 21, wherein the sample is from a mammal suspected of having MMP induced cancer.

23. The method of claim 22, wherein the mammal is a human.

24. An isolated nucleic acid comprising a sequence set forth in SEQ ID NOS: 1, 2, 3, or 4.


SEQUENCE LISTING (12 sequences)

SEQ ID NO: 1 - RNA, 19 nt, Artificial
  synthesized siRNA
cacaugugguaaagauaga

SEQ ID NO: 2 - RNA, 23 nt, Artificial
  synthesized siRNA
acaagccgauugccgacguguuc

SEQ ID NO: 3 - RNA, 25 nt, Artificial
  synthesized siRNA
gacaguuggagacacaugugguaaa

SEQ ID NO: 4 - RNA, 25 nt, Artificial
  synthesized siRNA
uggagacacaugugguaaagauaga

SEQ ID NO: 5 - DNA, 57 bp, Homo sapiens
ttggagacacatgtggtaaagatagaccctcgaggggcaaagacaagccgattgccg

SEQ ID NO: 6 - protein, 18 aa, Homo sapiens
VGDTCGKDRPSRGKDKPI

SEQ ID NO: 7 - DNA, 2355 bp, Homo sapiens
gggaggccggatgtgagtggagcggccatttcctgtttctctgcagttttcctcagctttgggtggtggccgctgccgggcatcggcttccagtccgcggagggcgaggcggcgtggacagcggccccggcacccagcgccccgccgcccgcaagccgcgcgcccgtccgccgcgccccgagcccgccgcttcctatctcagcgccctgccgccgccgccgcggcccagcgagcggccctgatgcaggccatcaagtgtgtggtggtgggagacggagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaaatgtctcagcccctcgttcttggtcctgtcccttggaacctttgtacgctttgctcaaaaaaaaacaaaaaaaaaaaacaaaaaaaaaaaacaacggtggagccttcgcactcaatgccaactttttgttacagattaatttttccataaaaccattttttgaaccaatcagtaattttaaggttttgtttgttctaaatgtaagagttcagactcacattctattaaaatttagccctaaaatgacaagccttcttaaagccttatttttcaaaagcgccccccccattcttgttcagattaagagttgccaaaataccttctgaactacactgcattgttgtgccgagaacaccgagcactgaactttgcaaagaccttcgtctttgagaagacggtagcttctgcagttaggaggtgcagacacttgctctcctatgtagttctcagatgcgtaaagcagaacagcctcccgaatgaagcgttgccattgaactcaccagtgagttagcagcacgtgttcccgacataacattgtactgtaatggagtgagcgtagcagctcagctctttggatcagtctttgtgatttcatagcgagttttctgaccagcttttgcggagattttgaacagaactgctatttcctctaatgaagaattctgtttagctgtgggtgtgccgggtggggtgtgtgtgatcaaaggacaaagacagtattttgacaaaatacgaagtggagatttacactacattgtacaaggaatgaaagtgtcacgggtaaaaactctaaaaggttaatttctgtcaaatgcagtagatgatgaaagaaaggttggtattatcaggaaatgttttcttaagcttttcctttctcttacacctgccatgcctccccaaattgggcatttaattcatctttaaactggttgttctgttagtcgctaacttagtaagtgcttttcttatagaaccccttctgactgagcaatatgcctccttgtattataaaatctttctgataatgcattagaaggtttttttgtcgattagtaaaagtgctttccatgttactttattcagagctaataagtgctttccttagttttctagtaactaggtgtaaaaatcatgtgttgcagctttatagtttttaaaatattttagataattcttaaactatgaaccttcttaacatcactgtcttgccagattaccgacactgtcacttgaccaatactgaccctctttacctcgcccacgcggacacacgcctcctgtagtcgctttgcctattgatgttcctttgggtctgtgaggttctgtaaactgtgctagtgctgacgatgttctgtacaacttaactcactggcgagaatacagcgtgggacccttcagccactacaacagaattttttaaattgacagttgcagaattgtggagtgtttttacattgatcttttgctaatgcaattagcattatgttttgcatgtatgacttaataaatccttgaatcataaaaaaaaaaaaaaa

SEQ ID NO: 8 - DNA, 2412 bp, Homo sapiens
gggaggccggatgtgagtggagcggccatttcctgtttctctgcagttttcctcagctttgggtggtggccgctgccgggcatcggcttccagtccgcggagggcgaggcggcgtggacagcggccccggcacccagcgccccgccgcccgcaagccgcgcgcccgtccgccgcgccccgagcccgccgcttcctatctcagcgccctgccgccgccgccgcggcccagcgagcggccctgatgcaggccatcaagtgtgtggtggtgggagacggagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagttggagaaacgtacggtaaggatataacctcccggggcaaagacaagccgattgccgatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaaatgtctcagcccctcgttcttggtcctgtcccttggaacctttgtacgctttgctcaaaaaaaaacaaaaaaaaaaaacaaaaaaaaaaaacaacggtggagccttcgcactcaatgccaactttttgttacagattaatttttccataaaaccattttttgaaccaatcagtaattttaaggttttgtttgttctaaatgtaagagttcagactcacattctattaaaatttagccctaaaatgacaagccttcttaaagccttatttttcaaaagcgccccccccattcttgttcagattaagagttgccaaaataccttctgaactacactgcattgttgtgccgagaacaccgagcactgaactttgcaaagaccttcgtctttgagaagacggtagcttctgcagttaggaggtgcagacacttgctctcctatgtagttctcagatgcgtaaagcagaacagcctcccgaatgaagcgttgccattgaactcaccagtgagttagcagcacgtgttcccgacataacattgtactgtaatggagtgagcgtagcagctcagctctttggatcagtctttgtgatttcatagcgagttttctgaccagcttttgcggagattttgaacagaactgctatttcctctaatgaagaattctgtttagctgtgggtgtgccgggtggggtgtgtgtgatcaaaggacaaagacagtattttgacaaaatacgaagtggagatttacactacattgtacaaggaatgaaagtgtcacgggtaaaaactctaaaaggttaatttctgtcaaatgcagtagatgatgaaagaaaggttggtattatcaggaaatgttttcttaagcttttcctttctcttacacctgccatgcctccccaaattgggcatttaattcatctttaaactggttgttctgttagtcgctaacttagtaagtgcttttcttatagaaccccttctgactgagcaatatgcctccttgtattataaaatctttctgataatgcattagaaggtttttttgtcgattagtaaaagtgctttccatgttactttattcagagctaataagtgctttccttagttttctagtaactaggtgtaaaaatcatgtgttgcagctttatagtttttaaaatattttagataattcttaaactatgaaccttcttaacatcactgtcttgccagattaccgacactgtcacttgaccaatactgaccctctttacctcgcccacgcggacacacgcctcctgtagtcgctttgcctattgatgttcctttgggtctgtgaggttctgtaaactgtgctagtgctgacgatgttctgtacaacttaactcactggcgagaatacagcgtgggacccttcagccactacaacagaattttttaaattgacagttgcagaattgtggagtgtttttacattgatcttttgctaatgcaattagcattatgttttgcatgtatgacttaataaatccttgaatcataaaaaaaaaaaaaaa

SEQ ID NO: 9 - protein, 15 aa, Homo sapiens
  MISC_FEATURE (1)..(1): Ac end
CGKDRPSRGKDKPIA

SEQ ID NO: 10 - protein, 6 aa, Artificial
  Factor Xa eymatic cleavage sequence
MHIEGR

SEQ ID NO: 11 - DNA, 24 bp, Artificial
  Synthetic blocking oligonucleotide
gaactagtngatcccccgggtcgc

SEQ ID NO: 12 - DNA, 23 bp, Artificial
  Synthetic control oligonucleotide
gaactagtgatcccccgggtcgc